(19) United States
(12) Patent Application Publication
(10) Pub. No.: US 2017/0088828 A1
(43) Pub. Date: Mar. 30, 2017

(54) COMPOSITIONS AND METHODS FOR TREATMENT OF LATENT VIRAL INFECTIONS

(71) Applicant: Agenovir Corporation, South San Francisco, CA (US)

(72) Inventor: Stephen R. Quake, Stanford, CA (US)

(21) Appl. No.: 15/277,578

(22) Filed: Sep. 27, 2016

Related U.S. Application Data
(60) Provisional application No. 62/234,347, filed on Sep. 29, 2015.

(51) Int. Cl. C12N 9/22

(52) U.S. Cl. CPC C12N 9/22

(57) ABSTRACT

Methods for treating latent viral infections using a gene for a nuclease that is expressed in the presence of a latent viral infection, allowing the nuclease to digest viral nucleic acid. The gene is controlled by a switch that turns expression on in the presence of viral transcripts. The switch may be an engineered sequence that, in the absence of a viral transcript, forms a duplex structure to inhibit translation. The viral transcript hybridizes to the switch and disrupts the duplex structure, allowing translation to occur. A nucleic acid encodes a nuclease and a switch that causes the nuclease to be expressed in the presence of a viral nucleic acid. A portion of the switch may be complementary to at least a portion of a latency associated transcript such as an HHV latency associated transcript that, when present, interacts with the switch to initiate translation of the nuclease.

### RELATED APPLICATION

This application claims the benefit of and priority to U.S. Provisional Patent Application Ser. No. 62/234,347, filed Sep. 29, 2015, incorporated by reference.

### TECHNICAL FIELD

The invention relates to treating viral infections using compositions that provide a nuclease to digest viral nucleic acid in the presence of latency-associated viral transcripts.

### BACKGROUND

Viral infections pose a significant medical problem. For example, herpes is a widespread human pathogen, with more than 90% of adults having been infected. Due to latency, once infected, a host carries the herpes virus indefinitely, even when not expressing symptoms. Similarly, human papillomavirus, or HPV, is a common virus in the human population, in which greater than 75% of people will be infected. A particular problem is that some viral infections may lead to cancer. For example, integration of HPV into host DNA is known to result in cancer, specifically cervical cancer. The Epstein-Barr virus (EBV) not only causes infectious mononucleosis (glandular fever), but is also associated with cancers such as Hodgkin's lymphoma and Burkitt's lymphoma.

Efforts are made to develop drugs that target viral proteins but those efforts have not been wholly successful. For example, when a virus is in a latent state, not actively expressing its proteins, there is no protein to target. Additionally, any effort to eradicate a viral infection is not optimal if it interferes with host cellular function. For example, an enzyme that prevents viral replication is not helpful if it interferes with genome replication in cells throughout the host.

### SUMMARY

The invention provides methods for treating latent viral infections by providing a gene for a nuclease that is expressed in the presence of a latent viral infection, allowing the nuclease to digest viral nucleic acid. The gene is accompanied by a switch that turns expression on in the presence of latent viral transcripts. The switch may be an engineered sequence around or near a ribosome binding site (RBS) or start codon for the gene, wherein in the absence of a latent viral transcript, the sequence forms a duplex structure that inhibits translation of the gene. A latent viral transcript acts as a trigger that hybridizes to the switch and disrupts the duplex structure, allowing translation to occur. The gene and the switch can be provided as DNA in, for example, a plasmid that gets transcribed into RNA in the infected tissue or cell(s); or may be provided in the RNA form. The RNA includes the switch, e.g., a riboswitch, engineered into its sequence at or around the RBS or start codon. The riboswitch may be designed to provide a latency-associated transcript with a “toehold” sequence to amplify dynamic range of expression—i.e., in the presence of the latency-associated transcript, expression is many-fold higher than the absence of the transcript. By targeting different regions of one or more different latency-associated transcripts, multiple switches can be provided that are orthogonal to one another—i.e., each amplifies expression in the presence of its respective trigger without crosstalk between switches and triggers.

Since the nuclease is expressed in the presence of a viral transcript, the viral nucleic acid is susceptible to digestion by the nuclease. The viral transcript interacts with the switch causing the encoded nuclease to be translated into the active enzyme. The nuclease then digests the viral nucleic acid. The nuclease is preferably a programmable nuclease. The nuclease can be, for example, a zinc finger nuclease, a meganuclease, a TALENs, Cpf1, PfAgo, or NgAgo, and is preferably Cas9, encoded along with a guide RNA that specifically targets the viral nucleic acid. Since the nuclease is only expressed in the presence of a viral transcript, possibility of interaction with non-target DNA is minimized. Since the switch can be engineered to be activated by a latency-associated transcript, the nuclease can be specifically activated in tissue or cells subject to a latent viral infection. The nuclease may be encoded by nucleic acid such as a plasmid and be delivered to target tissue through the use of a carrier such as a cationic lipid or polymer complex or with the application of an aid such as ultrasound, microneedles or electroporation. Thus, compositions of the invention can be delivered to local reservoirs of latent infection and used to digest the genome of the latent virus.

In certain aspects, the invention provides a nucleic acid that encodes a nuclease and a switch that causes the nuclease to be expressed in the presence of a viral nucleic acid. A portion of the switch may be complementary to at least a portion of a latency-associated transcript such as an HHV latency-associated transcript or a latency-associated transcript of pseudorabies virus. In some embodiments, the latency associated transcript, when present, interacts with the switch to initiate translation of the nuclease. The nucleic acid may be provided as a plasmid. The nucleic acid may be provided as mRNA including a 5 prime cap and a poly(A) tail. The nuclease may be Cas9 endonuclease and the nucleic acid may also encode a guide sequence that targets the nuclease to a target on a genome of a virus. The target may comprise a segment of at least 18 nucleotides that is at least 60% complementary to the guide sequence and is adjacent a protospacer adjacent motif (PAM), and wherein the target is not found in the host genome. For example, the target may include a portion of a genome or gene of one selected from the group consisting of: a hepatitis virus; a hepatitis B virus (HBV); an Epstein-Barr virus; a Kaposi's sarcoma-associated herpesvirus (KSHV); a herpes-simplex virus (HSV); a cytomegalovirus (CMV); and a human papilloma virus (HPV). Preferably, the switch is a riboswitch, e.g., a portion of the nucleic acid that, when transcribed into mRNA, forms a double stranded structure that blocks translation in the absence of the viral nucleic acid.

The switch may include one or more of a ribosome binding site and a start codon. In certain embodiments, the switch includes, i.e., at least partially spans or covers, one or more of a ribosome binding site and a start codon for the nuclease gene. Where the nuclease is Cas9, when the plasmid is transcribed into RNA and the latency associated transcript hybridizes to the riboswitch, the Cas9 endonuclease is expressed.

The invention may further include a carrier for delivering the nucleic acid (e.g., the plasmid) to cells in a subject. Suitable carriers include one or more of a liposome, a nanoparticle, a peptide, a polymer, a lipid, a cationic lipid complex, a cationic polymer complex, and a nanoplex.

In preferred embodiments, the viral nucleic acid required for expression of the nuclease is a latency-associated transcript. The nuclease may be a zinc-finger nuclease, a transcription activator-like effector nuclease, or a meganuclease or may preferably be a Cas9 nuclease.

In preferred embodiments, the nucleic acid further encodes a guide sequence that targets the nuclease to a target on a genome of a virus. For example, the nuclease may be Cas9 endonuclease and the guide sequence may be a guide RNA. The guide sequence preferably matches the target according to a predetermined criteria and does not match any portion of a host genome according to the predetermined criteria (e.g., is at least 60% complementary within a 20 nucleotide stretch and presence of a protospacer adjacent motif adjacent the 20 nucleotide stretch). The guide sequence should not match any portion of the host genome (e.g., human genome) according to the predetermined criteria.

Suitable targets in viral genomes include a portion of a genome or gene of a hepatitis virus, a hepatitis B virus (HBV), an Epstein-Barr virus, a Kaposi's sarcoma-associated herpesvirus (KSHV), a herpes-simplex virus (HSV), a cytomegalovirus (CMV), and a human papilloma virus (HPV). The target in the viral genome may lie within one or more of a preC promoter in a hepatitis B virus (HBV) genome, an S1 promoter in the HBV genome, an S2 promoter in the HBV genome, an X promoter in the HBV genome, a viral Cp (C promoter) in an Epstein-Barr virus genome, a minor transcript promoter region in a Kaposi's sarcoma-associated herpesvirus (KSHV) genome, a major transcript promoter in the KSHV genome, an Egr-1 promoter from a herpes-simplex virus (HSV), an ICP 4 promoter from HSV-1, an ICP 10 promoter from HSV-2, a cytomegalovirus (CMV) early enhancer element, a cytomegalovirus immediate-early promoter, an HPV early promoter, and an HPV late promoter.

In some embodiments, the switch causes translation of the nuclease upon hybridization of the viral nucleic acid to the switch. The nucleic acid may be DNA (e.g., a plasmid), such that the switch causes the nuclease to be expressed upon translation of the DNA into RNA and hybridization of the viral nucleic acid to the switch in the RNA. The DNA or plasmid may include features such as a nuclear localization signal, a promoter, or both.

The nucleic acid may be provided within a viral vector, i.e., the viral vector may encode a Cas9 endonuclease gene under the control of a riboswitch. In such aspects, the invention provides a viral vector encoding a gene for a nuclease (e.g., Cas9 endonuclease) and a riboswitch that controls expression of the gene in response to the presence of a trigger (e.g., a viral transcript, such as a latency associated transcript).

The riboswitch may be a portion of the nucleic acid that, when transcribed into mRNA, forms a double stranded structure that blocks translation in the absence of the viral nucleic acid. The viral nucleic acid, when present, may inhibit formation of the double stranded structure thus permitting translation of the nuclease.

In a circumstance in which the nucleic acid includes RNA, the switch may comprise RNA, a portion of which is complementary to at least a portion of a latency-associated transcript (e.g., an HHV latency associated transcript, a latency-associated transcript of pseudorabies virus, or others).

The provided nucleic acid may use the switch to cause expression of the nuclease in the presence of nucleic acid from any suitable virus including, but not limited to, adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, or banna virus.

In some aspects, the invention provides a pharmaceutical composition comprising any of the nucleic acids described above. The pharmaceutical composition may include a transfection-facilitating cationic lipid formulation. The pharmaceutical composition includes appropriate diluents, adjuvants, and carriers for delivering the active components to targeted cells. The carrier may be, for example, a liposome, a nanoparticle, a peptide, a polymer, a lipid, or a nanoplex. The formulation may include standard pharmacologic formulations, including timed release formulations and other well-known pharmaceutical formulations.

In related aspects, the invention provides for the use of any of the nucleic acids described above in the manufacture of a medicament for treatment of a viral infection, preferably a latent viral infection.

### BRIEF DESCRIPTION OF THE DRAWINGS

FIG. 1 shows a nucleic acid that encodes a nuclease and a switch that causes the nuclease to be expressed in the presence of a viral nucleic acid.

FIG. 2 is a diagram of an HHV genome.

FIG. 3 illustrates a type of riboswitch sometimes referred to as a riboregulator.

FIG. 4 shows a toehold riboswitch.

FIG. 5 shows a plasmid according to certain embodiments.

FIG. 6 illustrates gene delivery with an AAV vector.

FIG. 7 shows Cas9 endonuclease in a complex with a single guide RNA (sgRNA).

FIG. 8 describes an exemplary method for selecting a gRNA.

FIG. 9 outlines a similarity criteria for selecting a targeting sequence.

FIG. 10 shows a plasmid that includes a targeting sequence.

FIG. 11 diagrams treating a latent viral infection using a switched Cas9 gene.

FIG. 12 shows a cationic lipid complex.

FIG. 13 shows the HBV genome.

FIG. 14 shows a gel resulting from an in vitro CRISPR assay against HBV.

FIG. 15 shows a plasmid according to certain embodiments.

FIG. 16 diagrams the EBV genome.

FIG. 17 shows genomic context around guide RNA sgEBV2 and PCR primer locations.

FIG. 18 shows a large deletion induced by targeting sgEBV2.

FIG. 19 shows that sequencing confirmed the connection of expected cutting sites.

FIG. 20 shows a sequence from the HPV 18 viral genome along with various HPV 18 TALENs designed to bind multiple E6 gene segments.

FIG. 21 shows targeted regions of the HPV 18 E6 gene.

FIG. 22 shows viable cell counts for HPV 18+ HeLa cells transfected with plasmid DNA encoding certain TALEN and CRISPR/Cas9 complexes 5 days after transfection.

FIG. 23 shows a process for assessing the effect of a HPV 16-specific sgRNA and mRNA encoding Cas9 protein on HPV-16+ cells.

FIG. 24 shows normalized cell counts after 1, 3, and 6 days post-nucleofection with various Cas9 mRNA and sgRNA combinations.

FIG. 25 shows cell counts for cells treated with various sgRNA and a variety of Cas9 mRNA after 6 days.

FIG. 26 illustrates an HBV episomal DNA cell model.

FIG. 27 shows target locations on the HBV genome of various sgRNAs.

FIG. 28 shows results of gel electrophoresis separations indicating cleavage of HBV DNA in cells transduced with sgRT RNA, sgHBx RNA, sgCore RNA, and sgPreS1 RNA.

FIG. 29 shows HBV DNA quantity determined by qPCR in untreated cells and cells treated with HBV-specific sgRNAs and Cas9.

### DETAILED DESCRIPTION

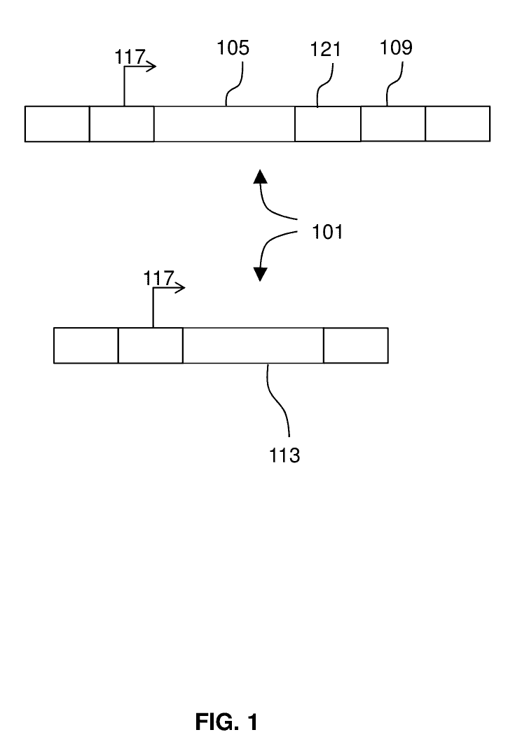

FIG. 1 shows a nucleic acid 101 that encodes a nuclease 105 and a switch 109 that causes the nuclease to be expressed in the presence of a viral nucleic acid. Other features may optionally be included in the nucleic acid 101. For example, the nucleic acid 101 may include a guide sequence 113 that targets the nuclease to a viral genomic target. The nucleic acid 101 may include one or more promoter 117 to aid in transcription of the included genes. Additionally, the nucleic acid 101 may include a portion that codes for a nuclear localization signal 121 so that the nuclease 105, when expressed by transcription and translation, is tagged for import into the nucleus of a host cell.

The switch 109 is a segment of nucleic acid 101 that, once nucleic acid 101 is in the RNA form (e.g., by transcription, where the nucleic acid 101 is provided as DNA), influences expression of the nuclease 105. In some embodiments, the switch is a segment of the RNA that forms a structure that inhibits translation in the absence of a viral nucleic acid. In that case, the viral nucleic acid and the switch include portions that are complementary to one another. The viral nucleic acid thus acts as a trigger for the switch by hybridizing via the complementary portions and changing the structure of the switch from one that inhibits translation to one that permits or initiates translation. In a preferred embodiment, the viral nucleic acid required for expression of the nuclease is a latency-associated transcript.

A latency-associated transcript (LAT) is a length of RNA that accumulates in cells hosting long-term, or latent, viral infections. The LAT RNA is produced by transcription from a specific region of the viral DNA. The LAT regulates the viral genome and may interfere with the normal activities of the infected host cell. Viruses known or suspected to exhibit LATs include viruses of the Herpesviridae family (e.g., Herpes simplex virus-1 (HSV-1), Herpes simplex virus-2 (HSV-2), Varicella zoster virus (VZV), Epstein-Barr virus (EBV), Cytomegalovirus (CMV), Roseolovirus, Herpes lymphotropic virus, Kaposi's sarcoma-associated herpesvirus (KSHV)) among others (e.g., pseudorabies virus). The nucleic acid that functions as a trigger may also be viral DNA that hybridizes to the trigger. In preferred embodiments, the trigger is a Human Herpes Virus Latency Associated Transcript (HHV LAT), transcribed from an HHV genome.

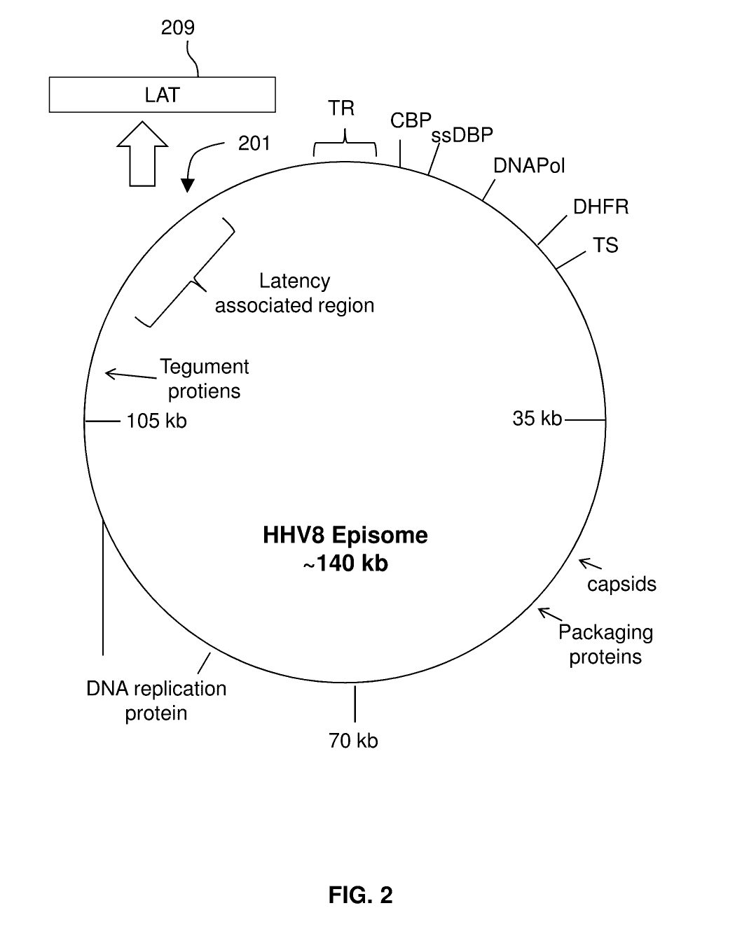

FIG. 2 is a diagram of parts of an HHV genome. Human cells having been infected with HHV-8 harbor multiple copies of the circularized genomes. As depicted, the circular episome represents a fusion of the terminal repeats (TR) at each end of the linear genome. The episome is approximately 140 kb in length and contains open reading frames that code for viral proteins that mediate latent infection as well as modulate cellular processes. Herpes virus may establish lifelong infection during which a reservoir virus population survives in host nerve cells for long periods of time. During the latent infection, the metabolism of the host cell is disrupted. While the infected cell would ordinarily undergo an organized death or be removed by the immune system, the consequences of LAT production interfere with those normal processes.

Latency is distinguished from lytic infection; in lytic infection many Herpes virus particles are produced and then burst or lyse the host cell. Lytic infection is sometimes known as “productive” infection. Latent cells harbor the virus for long time periods, then occasionally convert to productive infection which may lead to a recurrence of symptomatic Herpes symptoms. During latency, most of the Herpes DNA is inactive, with the exception of LAT, which accumulates within infected cells. The region of HHV DNA which encodes LAT is known as LAT-DNA. After splicing, LAT is a 2.0-kilobase transcript (or intron) produced from the 8.3-kb LAT-DNA. The DNA region containing LAT-DNA is known as the Latency Associated Transcript Region 201. The latency transcriptional unit 201 is transcribed into a LAT 209. The latency associated transcript 209 includes RNA that acts as a trigger for switch 109.

The switch 109 is a portion of the nucleic acid 101 that activates or deactivates expression of a gene. Any suitable switch 109 may be included. Preferably, the switch 109 is present in the RNA, e.g., where the nucleic acid 101 is provided as DNA (or example, in a plasmid), upon transcription of the DNA into RNA, the switch is provided by a segment of the RNA and may be referred to as a riboswitch.

### Riboswitch

A riboswitch is a regulatory segment of a messenger RNA molecule that interacts with a trigger, resulting in a change in production of the proteins encoded by the mRNA. The trigger may be a small molecule, crystal, metal, macromolecule such as nucleic acid, lipid, or protein, or other suitable particle. In some embodiments, the trigger may be small molecule metabolite. In preferred embodiments, the trigger is a nucleic acid such as the LAT 209. An mRNA that contains a riboswitch is directly involved in regulating its own activity, in response to the concentration or presence of the trigger.

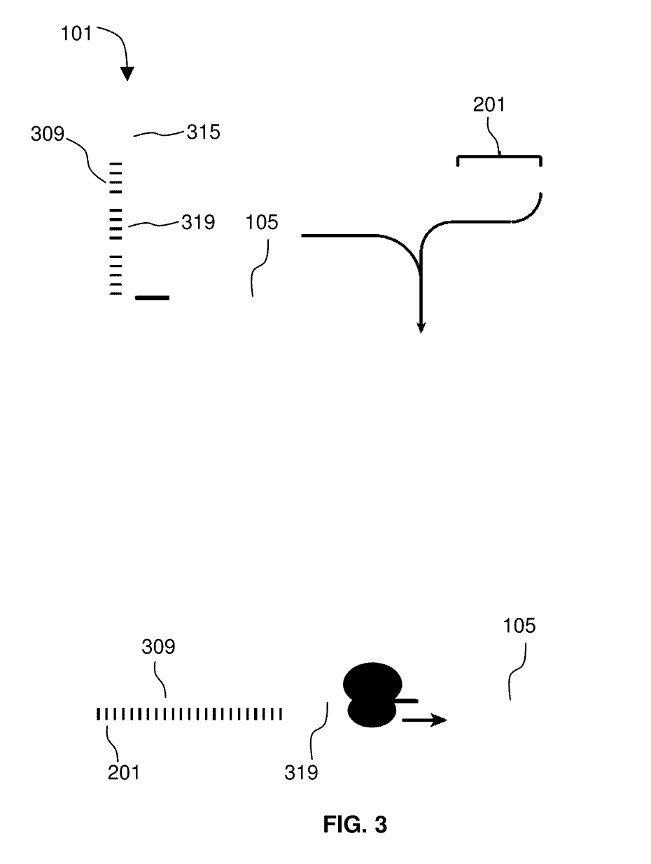

FIG. 3 illustrates the operation of one type of riboswitch 309, sometimes referred to as a conventional riboregulator. A riboregulator is a ribonucleic acid (RNA) that responds to a signal nucleic acid molecule by Watson-Crick base pairing. A riboregulator may respond to a signal, or trigger, molecule in any number of manners including, translation (or repression of translation) of the RNA into a protein, activation of a ribozyme, release of silencing RNA (siRNA), conformational change, and/or binding other nucleic acids. Riboregulators contain two canonical domains, a sensor domain and an effector domain. The sensor domain binds complementary RNA or DNA strands. Because binding is based on base-pairing, a riboregulator can be tailored to differentiate and respond to individual genetic sequences and combinations thereof.

Translational riboregulators regulate the ability of a ribosome complex to scan, assemble, or translate an RNA molecule into a protein. In translational riboregulators, the RNA molecule is repressed or de-repressed depending on the secondary structure of the RNA molecule. Signal-responsive structures are usually introduced into the 5′ untranslated region (5′ UTR) of the RNA molecules using standard molecular biological techniques. For translation, the small (40S) ribosome complex scans an RNA molecule from 5′ untranslated region to the start codon. When the complex encounters secondary structure, it must melt the structure to reach the start codon or it will fall off the molecule. The complex moves along through the untranslated region until it stalls just prior to reaching the start codon because it encounters a highly conserved sequence (a Kozak consensus sequence in eukaryotes). The stalled complex then combines with the large ribosome (60S) to begin translating the RNA into protein. As described in International Patent Application Publication No. WO 92/023070 to United States Biochemical Corporation (incorporated by reference), a riboswitch may use a self-pairing stem-loop that inhibits translation of RNA unless a complementary RNA sequence (anti-inhibitor) is present.

In alternative embodiments, a riboregulator may use antisense molecules to prevent translation. See, e.g., U.S. Pat. No. 6,323,003, incorporated by reference. In such systems, antisense molecules block translation unless removed via competitive hybridization and strand-displacement by specific signal RNA sequences. In certain embodiment, the switch 109 may be a translational riboregulator that responds to small molecules to function as a hybrid riboswitch/riboregulator molecule, termed an anti-switch. See Bayer & Smolke, 2005, Programmable ligand-controlled riboregulators of eurkaryotic gene expression, Nat Biotech 23(3):337-43, incorporated by reference. In an anti-switch, the presence of a small organic molecule binds an aptamer sequence in the RNA molecule which unmasks an otherwise sequestered antisense sequence, which can bind and block target RNA translation.

The riboregulator switch 309 of FIG. 3 includes an RNA molecule “transducer strand” that contains (from the 3′ to the 5′ end) a gene 105 for a nuclease, a start codon, a ribosome binding site (RBS) 319, and a YUNR loop 315 (Y for pyrimidine, N for any ribonucleotide, and R for purine). The YUNR sequence 315 specifies two intraloop hydrogen bonds forming a U-turn structure. This structure creates a sharp bend in the RNA phosphate-backbone and presents the following three to four bases in a solvent-exposed, stacked configuration providing a scaffold for rapid interaction with complementary RNA. The riboregulator riboswitch 309 is further defined by a cognate trans-activating RNA (taRNA) 201, or “trigger”.

In the absence of the taRNA 201, the riboregulator riboswitch 309 forms a stem-loop structure, stabilized by the YUNR sequence 315. The stem includes a homoduplex portion of the switch 309 that are engineered to be self-complementary. The ribosome binding site 319 is at least partially included the duplexed portions of the stem, making the ribosome binding site 319 unavailable to the 40S subunit, which prevents translation of the gene into the nuclease 105.

The taRNA 201, when present, hybridizes to the riboswitch 309, as encouraged by the three to four bases exposed by the YUNR sequence 315. Upon full hybridization, the homoduplex of the stem-loop structure is disrupted in favor of the switch/taRNA heteroduplex. Formation of the switch/taRNA duplex exposes the RBS 319, which allows the ribosome to assemble at the RBS and begin translation.

The sequence of the switch 309 can be engineered subject to only a few constraints (e.g., inclusion of the RBS). Moreover, it is possible and may be preferable to include a switch 109 that has fewer design constraints and greater performance such as a “toehold riboswitch”.

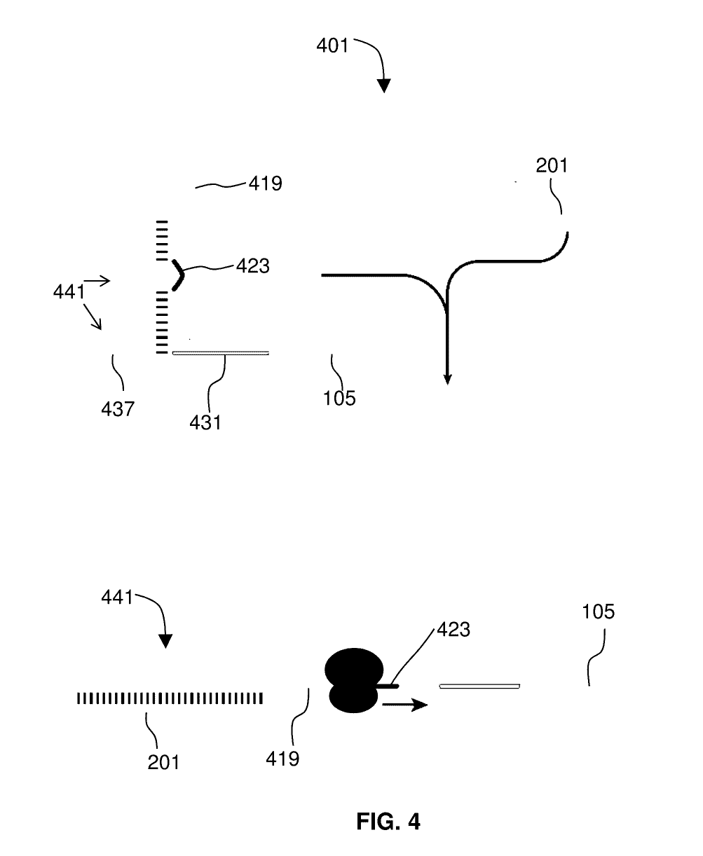

FIG. 4 shows a toehold riboswitch 401 for use in certain embodiments of the invention. The toehold riboswitch includes, in a 5′ to 3′ direction, a toehold 437, a trigger binding portion 441, an RBS 419, a start codon 423, a linker 431, and a gene 105 for the nuclease. The toehold switch 401 sequesters the region around the start codon to repress translation, rather than binding to either the RBS or the start codon. Instead of using loop regions to initiate interactions, the design exploits advantages afforded by linear-linear nucleic acid interaction and strand displacement. Interactions between strands are kinetically controlled through hairpins or multi-stranded complexes that feature the exposed single-stranded toehold 437. The toehold 437 serves as reaction initiation sites for the trigger and does not require a U-turn structure for accessibility. The toehold switch system uses two RNA strands referred to as the switch 401 and trigger 201. The switch RNA contains the coding sequence 105 of the gene being regulated. Upstream of this coding sequence is a hairpin-based processing module containing both a strong RBS 419 and a start codon 423 that is preferably followed by a common 21 nt linker sequence 431 coding for low-molecular-weight amino acids added to the N terminus of the gene of interest. A single-stranded toehold sequence 437 at the 5′ end of the hairpin module provides the initial binding site for the trigger RNA strand. This trigger molecule contains an extended single stranded region that completes a branch migration process with the hairpin to expose the RBS 419 and start codon 423, thereby initiating translation of the gene 105.

The hairpin processing unit functions as a repressor of translation in the absence of the trigger strand. Unlike other riboregulators, the RBS sequence is left completely unpaired within the 11 nt loop of the hairpin. The bases immediately before and after the start codon are sequestered within RNA duplexes that may be about 6 bp and 9 bp long, respectively. The start codon 423 is left unpaired, leaving a 3 nt bulge near the midpoint of the 18 nt hairpin stem. Due to the bulge, the cognate trigger strand in turn does not need to contain corresponding start codon bases, which allows for a great variety of trigger sequences. The 12 nt toehold domain at the 5′ end of the hairpin initiates interaction with the cognate trigger strand. The trigger RNA contains a 30 nt single-stranded RNA sequence that is complementary to the toehold and stem of the switch RNA. A toehold switch is described in Green et al., 2014, Toehold switches: de-novo-designed regulators of gene expression, Cell 159:925-939, incorporated by reference.

A switch can be included that activates translation in the presence of a certain transcript of interest. For the nucleic acid 101, translation is activated by the presence of a viral transcript, preferably a latency associated transcript such as an HHV latency associated transcript or a latency-associated transcript of pseudorabies virus. Thus, the switch causes translation of the nuclease 105 upon hybridization of the viral nucleic acid to the switch. As shown here, the riboswitch is a portion of the nucleic acid 101 that, when transcribed into mRNA, forms a double stranded structure that blocks translation in the absence of the viral nucleic acid. The viral nucleic acid when present inhibits formation of the double stranded structure thus permitting translation of the nuclease. The switch itself is RNA, a portion of which is complementary to at least a portion of a latency associated transcript. It can be seen that, where the nucleic acid 101 comprises DNA, the switch causes the nuclease to be expressed upon transcription of the DNA into RNA and hybridization of the viral nucleic acid to the switch in the RNA. The nucleic acid may further be provided in or as part of a vector, such as a viral or non-viral vector.

### Vectors

In some embodiments, the nucleic acid 101 is a non-viral vector. The gene 105 and the switch may be part of an expression cassette. A gene expression cassette typically includes a promoter 117 that drives transcription, the gene 105, and may include a termination signal to end gene transcription. Such an expression cassette can be embedded in a plasmid (circularized, double-stranded DNA molecule) as delivery vehicle.

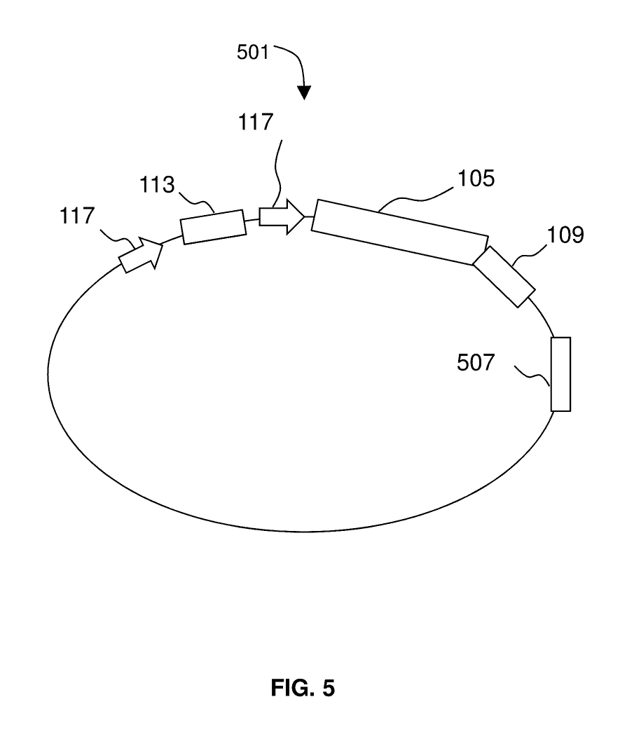

FIG. 5 shows a plasmid 501 according to certain embodiments. In the depicted embodiment, the plasmid 501 includes the nuclease gene 105 proximal the encoded switch 109. Where the nuclease is Cas9, the plasmid 501 may further include a guide sequence portion 113 that encodes a guide RNA. Those expressed segments may each or both be under the control of one or more promoters 117. Any suitable promoter may be used such as, for example, a U6 or H1 promoter or a viral promoter, and any one or multiple promoter can be constitutive or inducible. Plasmid 501 may also include within gene 105 a nuclear localization signal sequence such that the nuclease once translated has a peptide sequence that causes import into the nucleus.

The invention includes plasmids and methods of delivering plasmids. A plasmid may be directly injected in vivo by a variety of injection techniques, among which hydrodynamic injection achieves good gene transfer efficiency in major organs by quickly injecting a large volume of plasmid solution and temporarily inducing pores in cell membrane. See, e.g., Khorsandi, 2008, Cancer Gene Therapy 15:225-230 as well as U.S. Provisional Patent Application Ser. No. 62/142,192, filed Apr. 2, 2015, titled GENE DELIVERY METHODS AND COMPOSITIONS, and any U.S. patent or Pre-Grant Publication to publish from an application claiming priority to that provisional, each of which are incorporated by reference.

A plasmid 501 (e.g., with its negatively charged DNA) may be encouraged to penetrate hydrophobic cell membranes with a carrier such as a chemical or complex. Chemicals including cationic lipids and cationic polymers may be used to condense plasmid DNA into a lipoplex or polyplex, respectively. Those nanoparticles shield plasmid DNA from nuclease degradation in extracellular space and facilitate entry into target cells.

Following cellular uptake, the plasmid 501 may travel within an endosome. It may be desirable to avoid interference from elements such as the toll-like receptor 9 (which detects unmethylated CpG dinucleotides) by providing the nucleic acid 101 in a minicircle DNA (mcDNA) vector. The mcDNA differs from other plasmids in the lack of bacteria-derived, CpG-rich backbone sequences. When administered in vivo, mcDNA mediates safe, high, and sustainable transgene expression.

Where the plasmid 501 includes an expression cassette, at least some of that vector finds its way to the nucleus, where it may remain as non-integrating, episomal DNA and lead to transgene expression. A replication origin or a scaffold matrix attachment region (S/MAR) can be included in vector design to replicate and retain episomal DNA in daughter cells. S/MAR is a eukaryotic DNA sequence that attaches to the nuclear scaffold/matrix, and by doing so is capable of driving the replication of episomal DNA along with duplication of host genomic DNA during cell division.

DNA vectors such as the plasmid 501 may be preferable for their ease of scaled-up production, ability to carry large genes, and low immunotoxicity. For some applications and embodiment, it may be preferable to provide the nucleic acid 101 in a viral vector.

Viruses that infect mammals provide naturally evolved gene delivery vehicles for nucleic acids of the invention. The surface proteins on viral particles can interact with receptors on target cells, which triggers cellular uptake via endocytosis. Once inside a target cell, viral vectors deliver their genetic information in the form of DNA into the nucleus for viral gene expression. Viruses, such as human immunodeficiency virus (HIV), are among the most widely used in gene therapy. Replacing most of the viral genes with a therapeutic gene cassette, and retaining signal sequences that are essential for replication and packaging are strategies included in creating a viral vector. Any suitable viral vector can be used in the invention, including vectors based on gammaretrovirus, lentivirus, adenovirus (AdV), adeno-associated virus (AAV) and herpes simplex virus (HSV).

Gammaretrovirus and lentivirus are both retrovirus characterized by a RNA genome. They use a virus-derived reverse transcriptase and integrase to insert their proviral complementary DNA into the host genome. Gammaretrovirus transduces replicating cells, and lentivirus can transduce non-replicating cells. Vectors based on these two viruses may have envelope glycoproteins that are engineered for specific tissue or cell tropisms. For example, replacing the envelope glycoprotein with the G glycoprotein from vesicular stomatitis virus significantly increases vector stability (hence easier purification procedure and higher titers), and expands tropism to a wide range of cell types. For targeted gene delivery to a specific cell type, retroviral vectors can be pseudotyped with a viral glycoprotein that binds to a specific membrane receptor of that cell type. Furthermore, a viral glycoprotein can be fused with a ligand protein or antibody that recognizes cell type-specific surface molecules, providing a versatile way of cell type-specific gene delivery. Retroviral vectors are generally associated with integration into the host genome which ensures the stability of transgene and persistent transgene expression in daughter cells following genome replication and cell division.

In some embodiments, recombinant adenovirus (AdV) or adeno-associated virus (AAV) used. AdV contains a DNA genome that episomally resides in host nucleus. AdV is able to transduce a broad range of human cells. AAV includes a group of small single-stranded DNA viruses. Recombinant AAV (rAAV) vector carrying inverted terminal repeats as the only viral component may be used in certain embodiments. For rAAV vectors, it is largely the capsid that determines the tropism and transduction profile in different cell types.

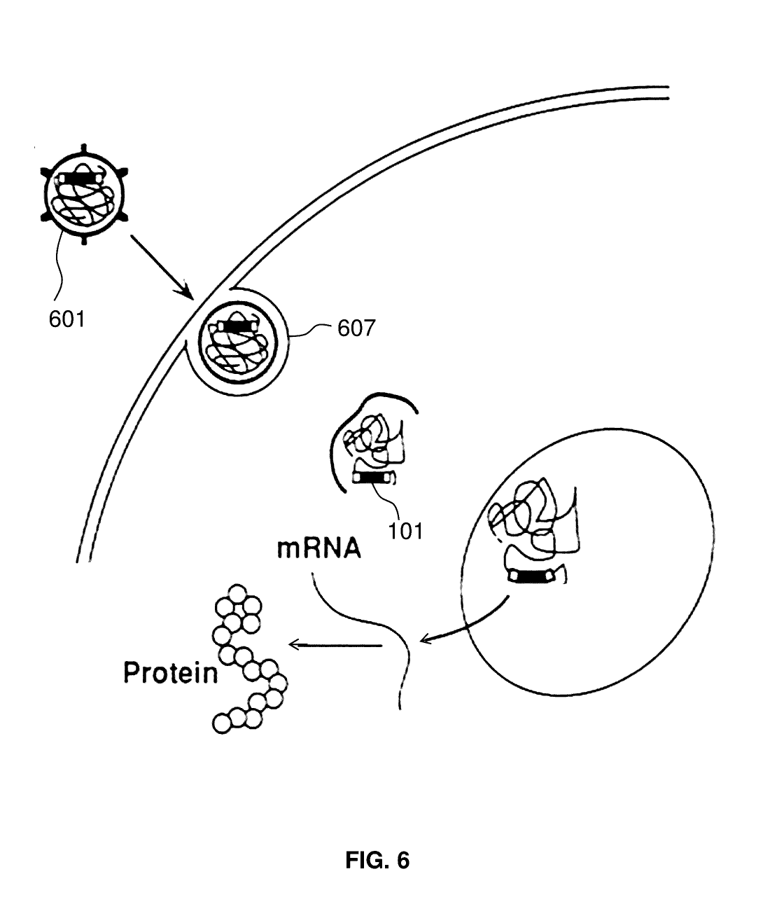

FIG. 6 illustrates gene delivery with an AAV vector. Using known methods, the nucleic acid is packaged in the adenovirus 601. The viral vector 601 fuses with the cell membrane by binding to adhesion molecules and becomes an endosome 607 within the lipid bi-layer. The vesicle opens in the cytoplasm, releasing the vector and the nucleic acid 101, which is transported to and enters the nucleus.

Vectors derived from some AAV serotypes such as AAV9 can cross the blood-brain barrier and transduce cells of the central nervous system (CNS) following a single intravenous injection. In addition to relying on natural diversity, AAV capsids can be decorated by peptides or “shuffled” to generate novel capsids that suit specific needs. For example, a chimeric AAV capsid “shuffled” from five parental natural AAV capsids was recently found to efficiently transduce human liver cells in a humanized mouse model (Lisowski et al., 2014, Nature 506:382). Similar to AdV vector, rAAV vector can transduce both dividing and non-dividing cells, and the recombinant viral genome stays in host nucleus predominantly as episome. Interestingly, single or multiple copies of rAAV vector genome can circularize in a head-to-tail or head-to-head configuration in host nucleus, thus enhancing stability of the episomal rAAV DNA genome and mediating long-term transgene.

An HSV vector may also be used. HSV is a naturally neurotropic virus. After initial infection in skin or mucous membranes, HSV is taken up by sensory nerve terminals, travels along nerves to neuronal cell bodies, and delivers its DNA genome into nuclei for replication. Therefore, HSV vectors are well suited for delivery to neurons.

However delivered, e.g., using a non-viral or a viral vector, the nucleic acid 101 includes a gene 105 for a nuclease.

### Nuclease

The invention provides nucleic acid compositions that encode a nuclease. In preferred embodiments, the invention is a composition that includes a nucleic acid that encodes nuclease under the control of a riboswitch. The nuclease is most preferably a programmable nuclease. The nucleic acid may be DNA (e.g., a plasmid or viral vector) or RNA (e.g., mRNA). If RNA, or if DNA, then once transcribed into RNA, the encoded programmable nuclease is under control of the riboswitch. Any suitable programmable nuclease may be used. The programmable nuclease may be an RNA-guided nuclease (e.g., a CRISPR-associated nuclease, such as Cas9 or a modified Cas9 or Cpf1 or modified Cpf1 or a homolog thereof). The programmable nuclease may be a TALEN or a modified TALEN. In certain embodiments, the programmable nuclease may be a DNA-guided nuclease (e.g., a Pyrococcus furiosus Argonaute (PfAgo) or Natronobacterium gregoryi Argonaute (NgAgo). The programmable nuclease may be a high-fidelity Cas9 (hi-fi Cas9), e.g., as described in Kleinstiver et al., 2016, High-fidelity CRISPR-Cas9 nucleases with no detectable genome-wide off-target effects, Nature 529:490-495, incorporated by reference.

In preferred embodiments, the programmable nuclease is Cas9, a nuclease that complexes with small RNAs as guides (gRNAs) to cleave DNA in a sequence-specific manner upstream of the protospacer adjacent motif (PAM) in any genomic location.

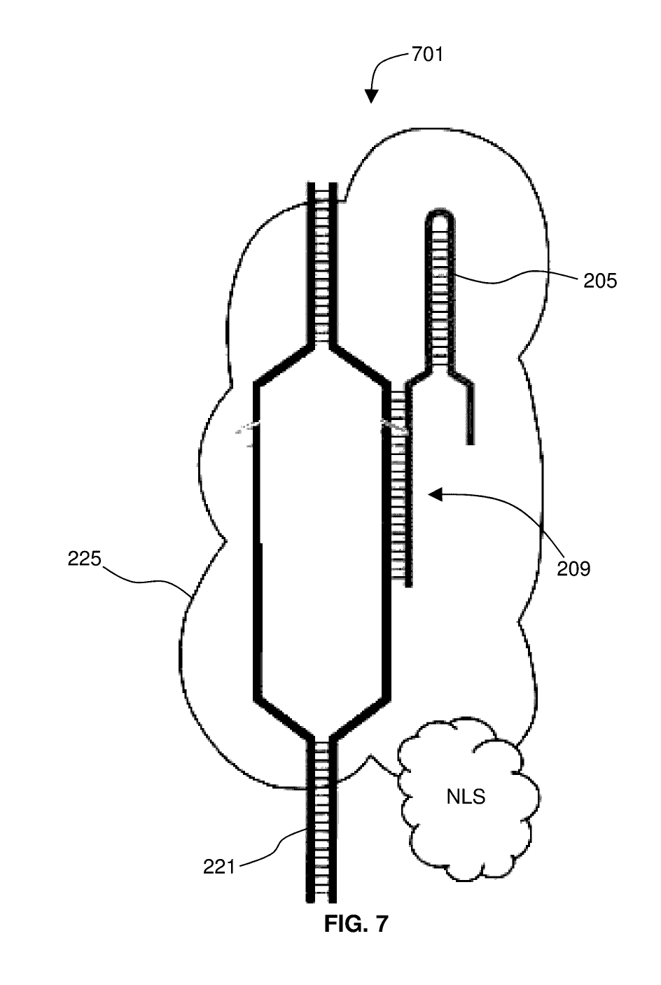

FIG. 7 shows a Cas9/gRNA complex 701 that includes a Cas9 endonuclease 725 in a complex with a single guide RNA (sgRNA) 705, bound to the target 721 viral genome via the guide sequence 709 of the guide RNA.

CRISPR may use separate guide RNAs known as the crRNA and tracrRNA. These two separate RNAs may be combined into a single RNA to enable site-specific genome cutting through the design of a short guide RNA. As used herein, guide RNA include any combination of sgRNA, crRNA, and tracrRNA used to guide Cas9 to the target. The Cas9 701 and guide RNA (gRNA) may be synthesized by known methods. Cas9/guide-RNA (gRNA) uses a nonspecific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11, Horvath et al., Science (2010) 327:167-170; Terns et al., Current Opinion in Microbiology (2011) 14:321-327; Bhaya et al. Annu Rev Genet (2011) 45:273-297; Wiedenheft et al. Nature (2012) 482:331-338); Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918), each incorporated by reference.

In an aspect of the invention, the Cas9 endonuclease causes a break at one or more locations in foreign nucleic acid. Two double strand breaks may cause a fragment of the genome to be deleted. Even if repair pathways anneal the two ends, there will still be a deletion in the genome. One or more deletions using the mechanism will incapacitate the viral genome.

In embodiments of the invention, nucleases cleave the genome of a target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, cleave only at very specific nucleotide sequences. In a preferred embodiment of the invention, the Cas9 nuclease is incorporated into the compositions and methods of the invention, however, it should be appreciated that any nuclease may be utilized.

In preferred embodiments of the invention, the Cas9 nuclease is used to cleave the genome. The Cas9 nuclease is capable of creating a double strand break in the genome. The Cas9 nuclease has two functional domains: RuvC and HNH, each cutting a different strand. When both of those domains are active, the Cas9 causes double strand breaks in the genome.

In some embodiments of the invention, insertions into the genome are designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the NHEJ repair pathway can be leveraged to disrupt the target gene. The changes introduced by the use of the CRISPR/gRNA/Cas9 system are permanent to the genome.

In some embodiments of the invention, at least one cut or insertion is caused by the nuclease. In a preferred embodiment, numerous cuts or insertions are caused in the genome, thereby incapacitating the virus. In an aspect of the invention, the number of insertions lowers the probability that the genome may be repaired.

In some embodiments of the invention, at least one deletion is caused by the gRNA/Cas9 complex. In a preferred embodiment, numerous deletions are caused in the genome, thereby incapacitating the virus. In an aspect of the invention, the number of deletions lowers the probability that the genome may be repaired. In a highly-preferred embodiment, the CRISPR/Cas9/gRNA system of the invention causes significant genomic disruption, resulting in effective destruction of the viral genome, while leaving the host genome intact.

TALENs uses a nonspecific DNA-cleaving nuclease fused to a DNA-binding domain that can be to target essentially any sequence. For TALEN technology, target sites are identified and expression vectors are made. Linearized expression vectors (e.g., by Not1) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55, incorporated by reference.

TALENs and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target. The breaks may be repaired via non-homologous end-joining or homologous recombination (HR). ZFN methods include introducing into the infected host cell nucleic acid 101 encoding a targeted ZFN and a switch as well as, optionally, at least one accessory polynucleotide. See, e.g., U.S. Pub. 2011/0023144 to Weinstein, incorporated by reference The cell includes target sequence. The cell is incubated to allow expression of the ZFN, wherein a double-stranded break is introduced into the targeted viral sequence by the ZFN. In some embodiments, a donor polynucleotide or exchange polynucleotide is introduced. Swapping a portion of the viral nucleic acid with irrelevant sequence can fully interfere transcription or replication of the viral nucleic acid. Target DNA along with exchange polynucleotide may be repaired by an error-prone non-homologous end-joining DNA repair process or a homology-directed DNA repair process.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. An engineered zinc finger binding domain may have a novel binding specificity compared to a naturally-occurring zinc finger protein. Engineering methods include, but are not limited to, rational design and various types of selection. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference.

A ZFN also includes a cleavage domain. The cleavage domain portion of the ZFNs may be obtained from any suitable endonuclease or exonuclease such as restriction endonucleases and homing endonucleases. See, for example, Belfort & Roberts, 1997, Homing endonucleases: keeping the house in order, Nucleic Acids Res 25(17):3379-3388. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction endonucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference.

In the ZFN-mediated process, a double stranded break introduced into the target sequence by the ZFN is repaired, via homologous recombination with the exchange polynucleotide, such that the sequence in the exchange polynucleotide may be exchanged with a portion of the target sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the target sequence. Thus, a portion of the viral nucleic acid may be converted to the sequence of the exchange polynucleotide. ZFN methods can include using a vector to deliver a nucleic acid molecule encoding a ZFN and, optionally, at least one exchange polynucleotide or at least one donor polynucleotide to the infected cell.

Meganucleases are endonucleases characterized by a large recognition site (sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs. Meganucleases have been found in all kingdoms of life, generally encoded within introns or inteins although freestanding members also exist. Crystal structures have illustrate mode of sequence specificity and cleavage mechanism for meganucleases: (i) specificity contacts arise from the burial of extended β-strands into the major groove of the DNA, with the DNA binding saddle having a pitch and contour mimicking the helical twist of the DNA; (ii) full hydrogen bonding potential between the protein and DNA is never fully realized; (iii) cleavage to generate the characteristic 4-nt 3′-OH overhangs occurs across the minor groove, wherein the scissile phosphate bonds are brought closer to the protein catalytic core by a distortion of the DNA in the central “4-base” region; (iv) cleavage occurs via a proposed two-metal mechanism, sometimes involving a unique “metal sharing” paradigm; (v) and finally, additional affinity and/or specificity contacts can arise from “adapted” scaffolds, in regions outside the core α/β fold. See Silva et al., 2011, Meganucleases and other tools for targeted genome engineering, Curr Gene Ther 11(1):11-27, incorporated by reference.

Some embodiments of the invention utilize a modified version of a nuclease. Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. Similar to the inactive dCas9 (RuvC- and HNH-), a Cas9 nickase is still able to bind DNA based on gRNA specificity, though nickases will only cut one of the DNA strands. The RuvC domain can be inactivated by a D10A mutation and the HNH domain can be inactivated by an H840A mutation.

A single-strand break, or nick, is normally quickly repaired through the HDR pathway, using the intact complementary DNA strand as the template. However, two proximal, opposite strand nicks introduced by a Cas9 nickase are treated as a double strand break, in what is often referred to as a ‘double nick’ or ‘dual nickase’ CRISPR system. A double-nick induced double strain break can be repaired by either NHEJ or HDR depending on the desired effect on the gene target. At these double strain breaks, insertions and deletions are caused by the CRISPR/Cas9 complex. In an aspect of the invention, a deletion is caused by positioning two double strand breaks proximate to one another, thereby causing a fragment of the genome to be deleted.

In some embodiments, a nuclease is a directed RNA nuclease that cleaves RNA from viruses or viral transcripts. One targetable RNA nuclease system is the Type III-A CRISPR-Cas Csm complex of Thermus thermophilus (TtCsm). TtCsm is composed of five different protein subunits (Csm1-Csm5) with an uneven stoichiometry and a single crRNA of variable size (35-53 nt). The TtCsm crRNA content is similar to the Type III-B Cmr complex, indicating that crRNAs are shared among different subtypes. TtCsm cleaves complementary target RNAs at multiple sites. Unlike Type I complexes, interference by TtCsm does not proceed via initial base pairing by a seed sequence. For discussion see Staals et al., 2014, RNA Targeting by the type III-A CRISPR-Cas Csm complex of Thermus thermophiles, Molecular Cell 56(4):518-530, incorporated by reference. Thus aspects of the invention provide a nucleic acid that encodes nuclease that can be activated to digest foreign RNA. The nuclease may be TtCsm or any other suitable targetable nuclease that cuts RNA.

In some embodiments, the invention includes the use of the Dicer, the RNA-induced silencing complex (RISC), or both. Dicer, also known as endoribonuclease Dicer or helicase with RNase motif, is an enzyme of the RNase III family. Dicer cleaves double-stranded RNA (dsRNA) and pre-microRNA (pre-miRNA) into short double-stranded RNA fragments called small interfering RNA and microRNA respectively. These fragments are approximately 20-25 base pairs long with a two-base overhang on the 3′ end. Dicer facilitates the activation of the RNA-induced silencing complex (RISC), which is essential for RNA interference. RISC has a catalytic component argonaute, which is an endonuclease capable of degrading messenger RNA (mRNA).

RISC is a multi-protein complex, specifically a ribonucleoprotein, which incorporates one strand of a double-stranded RNA (dsRNA) fragment, such as small interfering RNA (siRNA) or microRNA (miRNA). The single strand acts as a template for RISC to recognize complementary messenger RNA (mRNA) transcript. Once found, argonaute activates and cleaves the mRNA. This process is called RNA interference (RNAi) and provides for gene silencing and defense against viral infections.

The RNase III Dicer aids RISC in RNA interference by cleaving dsRNA into 21-23 nucleotide long fragments with a two-nucleotide 3′ overhang. These dsRNA fragments are loaded into RISC and each strand has a different fate based on the asymmetry rule phenomenon.

The strand with the less stable 5′ end is selected by the argonaute and integrated into RISC. This strand is known as the guide strand. The other strand, known as the passenger strand, is degraded by RISC. RISC uses the bound guide strand to target complementary 3′-untranslated regions (3′UTR) of mRNA transcripts via Watson-Crick base pairing. RISC can now regulate gene expression of the mRNA transcript in a number of ways. RISC degrades target mRNA which reduces the levels of transcript available to be translated by ribosomes. There are two main requirements for mRNA degradation to take place: a near-perfect complementary match between the guide strand and target mRNA sequence; and a catalytically active argonaute protein, called a ‘slicer’, to cleave the target mRNA. Also, RISC can modulate the loading of ribosome and accessory factors in translation to repress expression of the bound mRNA transcript. Translational repression only requires a partial sequence match between the guide strand and target mRNA. Translation can be regulated at the initiation step by preventing the binding of the eukaryotic translation initiation factor (eIF) to the 5′ cap. It has been noted RISC can adeadenylate the 3′ poly(A) tail which might contribute to repression via the 5′ cap. RISC may also prevent the binding of the 60S ribosomal subunit to the mRNA.

Argonaute proteins are a family of proteins that play a role in RNA silencing as a component of the RNA-induced silencing complex (RISC). The Argonaute of the archaeon Pyrococcus furiosus (PfAgo) uses small 5′-phosphorylated DNA guides to cleave both single stranded and double stranded DNA targets, and does not utilize RNA as guide or target.

NgAgo uses 5′ phosphorylated DNA guides (so called “gDNAs”) and appear to exhibit little preference for any certain guide sequences and thus may offer a general-purpose DNA-guided programmable nuclease. NgAgo does not require a PAM sequence, which contributes to flexibility in choosing a genomic target. NgAgo also appears to outperform Cas9 in GC-rich regions. NgAgo is only 887 amino acids in length. NgAgo randomly removes 1-20 nucleotides from the cleavage site specified by the gDNA. Thus, PfAgo and NgAgo represent potential DNA-guided programmable nucleases that may be modified for use as a composition of the invention.

A nucleic acid of the invention may encode a targeting nuclease that uses a targeting sequence such as a guide RNA (gRNA) to target and digest foreign nucleic acid while avoiding off-target (e.g., self) digestion. The invention provides methods to avoid self-genome digestion. A targeting sequence may be pre-determined (e.g., to protect against a specific virus) and encoded within the transgene.

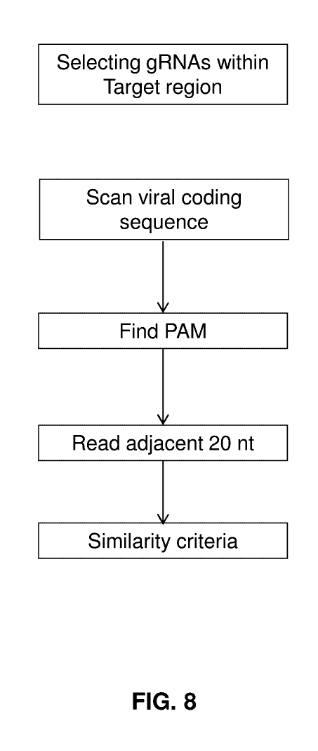

FIG. 8 describes an exemplary method for selecting a gRNA within the viral target region. A system or method of the invention may be used to scan the viral coding sequence and finds the PAM for the nuclease that is to be used. For example, where the digestion system will include cas9, the system scan the target for NGG, where N is any nucleotide. Upon finding the PAM in the viral genome, the 20 nucleotide string adjacent to the PAM within the viral genome are read. This 20 nucleotide string is provisionally treated as a potential sequence for the gRNA. Finally selecting the nucleotide string for the gRNA involves determining if the nucleotide string satisfies a similarity criteria for any region within the host genome (i.e., a gRNA is only selected if there is no region within the host genome that is similar according to a defined criteria).

Any suitable similarity criteria may be used. For example, one similarity criteria may be the requirement of a perfect match for all 20 bases of the nucleotide string. Other criteria may include that 19 bases match, or 18, etc. In a preferred embodiment, the invention includes similarity criteria that balance the requirement of actually finding a useful gRNA with the probabilities of some matching portions in the host, i.e., the possibility that even without a perfect 20 nt match, some of the gRNA may still bind to the host genome and initiate nuclease action. The includes similarity criteria that minimize off-target action against the host genome.

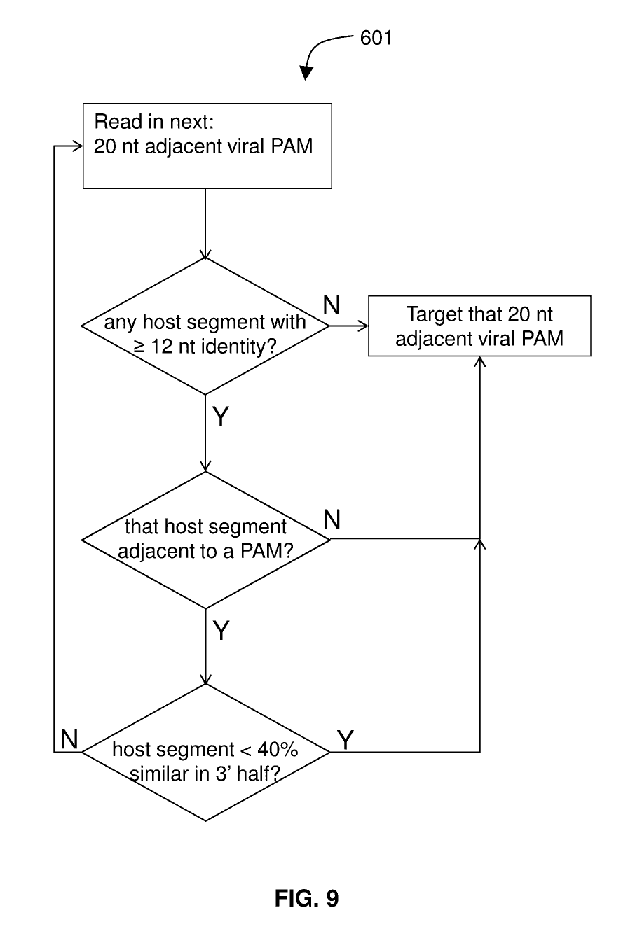

FIG. 9 outlines similarity criteria 601 according to certain embodiments that may be automatically applied by, for example, a computer system. To avoid digestion of host genome, the system applies a search criteria that embodies certain principles. The system preferably tries to avoid any target sequence with any approximately 12 nt DNA stretch homology to the human genome. When homology to human genome is inevitable, the guide RNA candidate not followed by PAM in the human genome would not lead to off-target digestion, and should be given priority. If homologous sequences and PAM both are present in the human genome, one should choose the guide RNA candidate with low homology (e.g., <40% similar) to human genome in the half next to PAM, where double strand break happens.

To reach these principles, as diagrammed in FIG. 9, the system reads in a 20 nucleotide nucleotide string adjacent a PAM in the viral sequence. The system examines the host genome for any segment with ≧12 nucleotide identity to the nucleotide string. If no such segment is found (N), then that nucleotide string is provided as the guide sequence to target that 20 nucleotide in the viral genome. If such a segment is found in the human genome (Y), then the system determines if that segment in the host genome is adjacent to a PAM. If that segment in the host genome is not adjacent to a PAM (N), then that nucleotide string is provided as the guide sequence to target that 20 nucleotide in the viral genome. If that segment in the host genome is adjacent to a PAM (Y), then the system determines if the half of that segment that is closest to the PAM is less than 40% similar to the nucleotide string. If the half of that segment that is closest to the PAM is less than 40% similar to the nucleotide string (Y), then that nucleotide string is provided as the guide sequence to target that 20 nt in the viral genome. If the half of that segment that is closest to the PAM is not less than 40% similar to the nucleotide string, then the system reads in the next 20 nt nucleotide string in the viral genome sequence that is adjacent to a PAM and repeats the steps on that next candidate string. The cycle of steps is optionally repeated until at least one guide sequence is provided. Optionally, the steps may be repeated until several or all possible guide sequences are provided. The selected sequences are then included in a nucleic acid of the invention.

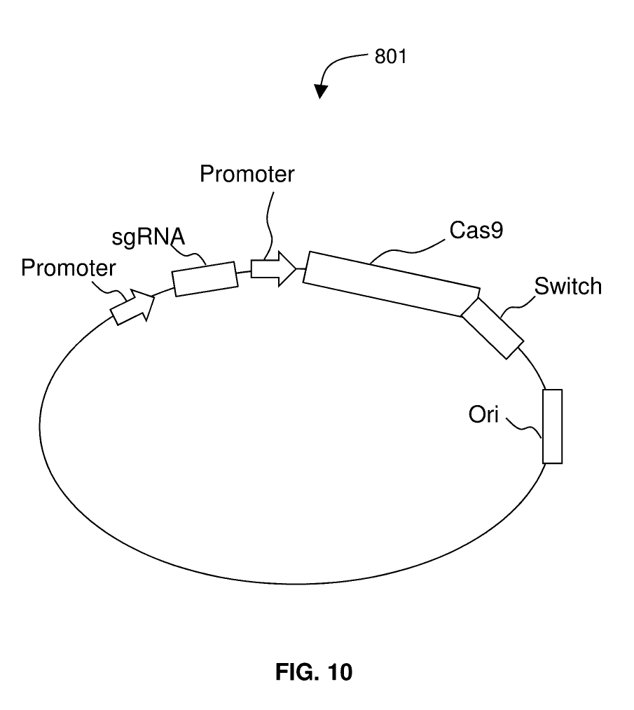

FIG. 10 shows a plasmid of the invention that includes a targeting sequence. A targeting sequence that satisfies the requirements described above may be included, e.g., as part of the stretch labeled sgRNA. More particularly, the plasmid will directly include as DNA the reverse complement of the single guide RNA, a portion of which is the targeting sequence. Such a nucleic acid provides a method for treating a latent viral infection.

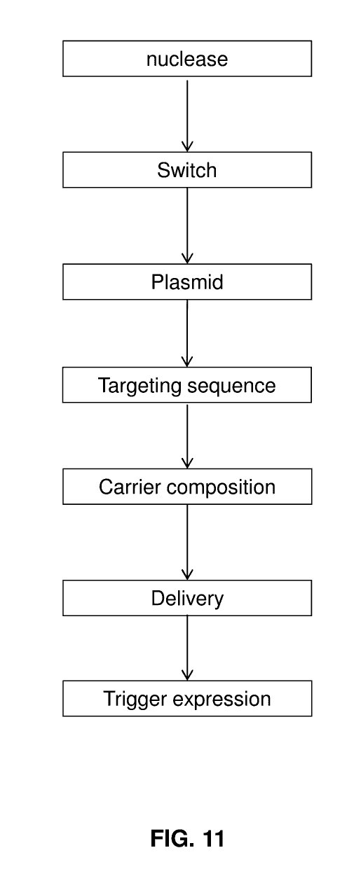

FIG. 11 diagrams a method for treating a latent viral infection using a switched Cas9 gene. The method includes obtaining a plasmid that includes a gene for a nuclease and a switch. The plasmid may also include determining and including a suitable targeting sequence (e.g., by analyzing a viral genome and a host genome to identify a suitable target in the viral genome that matches the targeting sequence according to a similarity criteria and does not so match any portion of the host genome). The plasmid is provided with a suitable carrier composition such as a cationic complex—e.g., a cationic polymer complex or a cationic lipid complex. some embodiments, the nucleic acid 101 is provided as part of a pharmaceutical composition or carriers. Compositions of the invention may be delivered by any suitable method include subcutaneously, transdermally, by hydrodynamic gene delivery, topically, or any other suitable method. In some embodiments, the nucleic acid 101 is provided in a carrier that is suitable for topical application to the human skin. The nucleic acid may be delivered to a cell or tissue in situ by delivery to tissue in a host. Introducing the composition into the host cell may include delivering the composition non-systemically to a local reservoir of the viral infection in the host, for example, topically.

A nucleic acid of the invention may be delivered to the affected area of the skin in a acceptable topical carrier such as any acceptable formulation that can be applied to the skin surface for topical, dermal, intradermal, or transdermal delivery of a medicament. The combination of an acceptable topical carrier and the compositions described herein is termed a topical formulation of the invention. Topical formulations of the invention are prepared by mixing the composition with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed.); Ghosh, T. K.; et al. TRANSDERMAL AND TOPICAL DRUG DELIVERY SYSTEMS (1997).

Topical carriers useful for topical delivery of compounds described herein may be any carrier known in the art for topically administering pharmaceuticals, for example, but not limited to, acceptable solvents, such as a polyalcohol or water; emulsions (either oil-in-water or water-in-oil emulsions), such as creams or lotions; micro emulsions; gels; ointments; liposomes; powders; and aqueous solutions or suspensions, such as standard ophthalmic preparations.

In certain embodiments, the topical carrier used to deliver the compositions described herein is an emulsion, gel, or ointment. Emulsions, such as creams and lotions are suitable topical formulations for use in accordance with the invention. An emulsion is a dispersed system comprising at least two immiscible phases, one phase dispersed in the other as droplets ranging in diameter from 0.1 μm to 100 μm. An emulsifying agent is typically included to improve stability.

In another embodiment, the topical carrier is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995). Other suitable gels for use in the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002). Polymer thickeners (gelling agents) that may be used include those known to one skilled in the art, such as hydrophilic and hydro-alcoholic gelling agents frequently used in the cosmetic and pharmaceutical industries. Preferably the gelling agent comprises between about 0.2% to about 4% by weight of the composition. The agent may be a cross-linked acrylic acid polymers that are given the general adopted name carbomer. These polymers dissolve in water and form a clear or slightly hazy gel upon neutralization with a caustic material such as sodium hydroxide, potassium hydroxide, or other amine bases.

In another preferred embodiment, the topical carrier is an ointment. Ointments are oleaginous semisolids that contain little if any water. Preferably, the ointment is hydrocarbon based, such as a wax, petrolatum, or gelled mineral oil.

In another embodiment, the topical carrier used in the topical formulations of the invention is an aqueous solution or suspension, preferably, an aqueous solution. Well-known ophthalmic solutions and suspensions are suitable topical carriers for use in the invention. The pH of the aqueous topical formulations of the invention are preferably within the range of from about 6 to about 8. To stabilize the pH, preferably, an effective amount of a buffer is included. In one embodiment, the buffering agent is present in the aqueous topical formulation in an amount of from about 0.05 to about 1 weight percent of the formulation. Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation. Preferably, the aqueous topical formulations of the invention have a viscosity in the range of from 0.015 to 0.025 Pa·s (about 15 cps to about 25 cps). The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

The topical formulations of the invention can include acceptable excipients such as protectives, adsorbents, demulcents, emollients, preservatives, antioxidants, moisturizers, buffering agents, solubilizing agents, skin-penetration agents, and surfactants. Suitable protectives and adsorbents include, but are not limited to, dusting powders, zinc sterate, collodion, dimethicone, silicones, zinc carbonate, aloe vera gel and other aloe products, vitamin E oil, allatoin, glycerin, petrolatum, and zinc oxide. Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate. Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin. Chlorine dioxide (ClO2), preferably, stabilized chlorine dioxide, is a preferred preservative for use with topical formulations of the invention. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use in the invention include, but are not limited to, acetate buffers, citrate buffers, phosphate buffers, lactic acid buffers, and borate buffers. Suitable solubilizing agents include, but are not limited to, quaternary ammonium chlorides, cyclodextrins, benzyl benzoate, lecithin, and polysorbates. Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

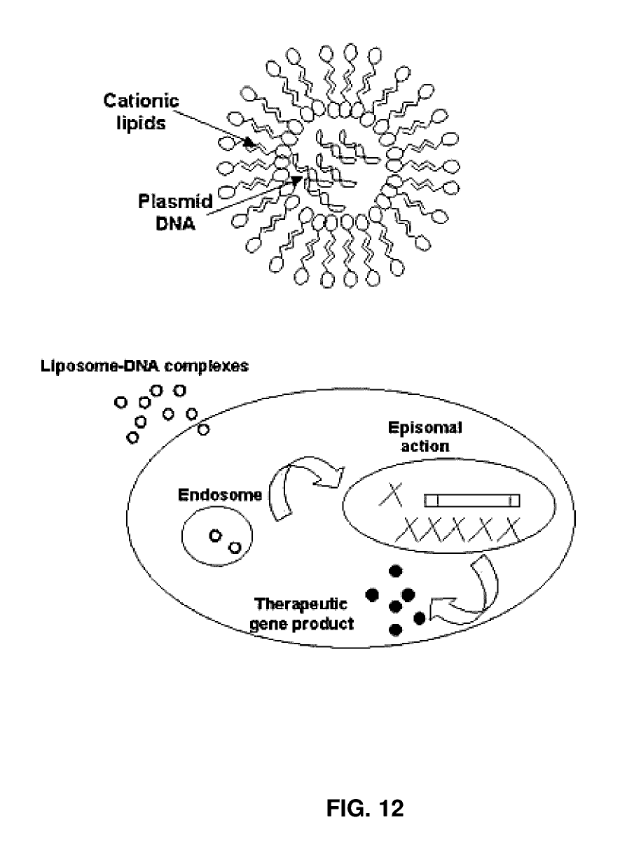

FIG. 12 shows a cationic lipid complex. Whatever other carriers are included, FIG. 12 illustrates the use of cationic lipids to create a lipo some for delivery (although other lipid complexes and compositions are within the scope of the invention) and delivery by liposome. Delivery may be effected through the use of a liposome, a nanoparticle, a peptide, a polymer, a lipid, or a nanoplex. Methods of the invention include using the nucleic acid 101 in the manufacture of a medicament for treatment of a viral infection. The medicament may include any of the suitable carriers such as a topical carrier and/or cationic complex. By delivering the nucleic acid to the target cell or tissue, methods of the invention may be used for the treatment of a viral infection by delivering a nucleic acid encoding a nuclease that is under the control of a switch that causes expression of the nuclease in the presence of viral nucleic acid. In some embodiments, methods of the invention are used to treat hepatitis B virus (HBV).

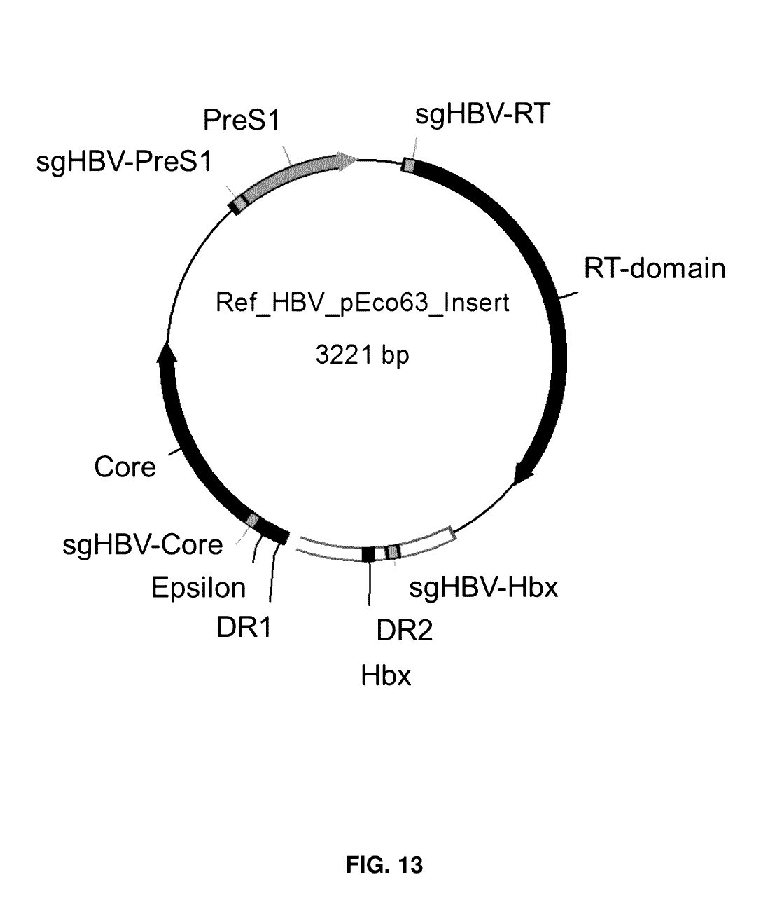

FIG. 13 diagrams the HBV genome. HBV, which is the prototype member of the family Hepadnaviridae, is a 42 nm partially double stranded DNA virus, composed of a 27 nm nucleocapsid core (HBcAg), surrounded by an outer lipoprotein coat (also called envelope) containing the surface antigen (HBsAg). The virus includes an enveloped virion containing 3 to 3.3 kb of relaxed circular, partially duplex DNA and virion-associated DNA-dependent polymerases that can repair the gap in the virion DNA template and has reverse transcriptase activities. HBV is a circular, partially double-stranded DNA virus of approximately 3200 bp with four overlapping ORFs encoding the polymerase (P), core (C), surface (S) and X proteins. During infection, viral nucleocapsids enter the cell and reach the nucleus, where the viral genome is delivered. In the nucleus, second-strand DNA synthesis is completed and the gaps in both strands are repaired to yield a covalently closed circular DNA molecule that serves as a template for transcription of four viral RNAs that are 3.5, 2.4, 2.1, and 0.7 kb long. These transcripts are polyadenylated and transported to the cytoplasm, where they are translated into the viral nucleocapsid and precore antigen (C, pre-C), polymerase (P), envelope L (large), M (medium), S (small)), and transcriptional transactivating proteins (X). The envelope proteins insert themselves as integral membrane proteins into the lipid membrane of the endoplasmic reticulum (ER). The 3.5 kb species, spanning the entire genome and termed pregenomic RNA (pgRNA), is packaged together with HBV polymerase and a protein kinase into core particles where it serves as a template for reverse transcription of negative-strand DNA. The RNA to DNA conversion takes place inside the particles.

Numbering of base pairs on the HBV genome is based on the cleavage site for the restriction enzyme EcoR1 or at homologous sites, if the EcoR1 site is absent. However, other methods of numbering are also used, based on the start codon of the core protein or on the first base of the RNA pregenome. Every base pair in the HBV genome is involved in encoding at least one of the HBV protein. However, the genome also contains genetic elements which regulate levels of transcription, determine the site of polyadenylation, and even mark a specific transcript for encapsidation into the nucleocapsid. The four ORFs lead to the transcription and translation of seven different HBV proteins through use of varying in-frame start codons. For example, the small hepatitis B surface protein is generated when a ribosome begins translation at the ATG at position 155 of the adw genome. The middle hepatitis B surface protein is generated when a ribosome begins at an upstream ATG at position 3211, resulting in the addition of 55 amino acids onto the 5′ end of the protein.

ORF P occupies the majority of the genome and encodes for the hepatitis B polymerase protein. ORF S encodes the three surface proteins. ORF C encodes both the hepatitis e and core protein. ORF X encodes the hepatitis B X protein. The HBV genome contains many important promoter and signal regions necessary for viral replication to occur. The four ORFs transcription are controlled by four promoter elements (preS1, preS2, core and X), and two enhancer elements (Enh I and Enh II). All HBV transcripts share a common adenylation signal located in the region spanning 1916-1921 in the genome. Resulting transcripts range from 3.5 nucleotides to 0.9 nucleotides in length. Due to the location of the core/pregenomic promoter, the polyadenylation site is differentially utilized. The polyadenylation site is a hexanucleotide sequence (TATAAA) as opposed to the canonical eukaryotic polyadenylation signal sequence (AATAAA). The TATAAA is known to work inefficiently, suitable for differential use by HBV.

There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in-frame start (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood but it is associated with the development of liver cancer. It stimulates genes that promote cell growth and inactivates growth regulating molecules.

HBV starts its infection cycle by binding to the host cells with PreS1. Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces.

HBV replicates its genome by reverse transcription of an RNA intermediate. The RNA templates is first converted into single-stranded DNA species (minus-strand DNA), which is subsequently used as templates for plus-strand DNA synthesis. DNA synthesis in HBV use RNA primers for plus-strand DNA synthesis, which predominantly initiate at internal locations on the single-stranded DNA. The primer is generated via an RNase H cleavage that is a sequence independent measurement from the 5′ end of the RNA template. This 18 nt RNA primer is annealed to the 3′ end of the minus-strand DNA with the 3′ end of the primer located within the 12 nt direct repeat, DR1. The majority of plus-strand DNA synthesis initiates from the 12 nt direct repeat, DR2, located near the other end of the minus-strand DNA as a result of primer translocation. The site of plus-strand priming has consequences. In situ priming results in a duplex linear (DL) DNA genome, whereas priming from DR2 can lead to the synthesis of a relaxed circular (RC) DNA genome following completion of a second template switch termed circularization. It remains unclear why hepadnaviruses have this added complexity for priming plus-strand DNA synthesis, but the mechanism of primer translocation is a potential therapeutic target. As viral replication is necessary for maintenance of the hepadnavirus (including the human pathogen, hepatitis B virus) chronic carrier state, understanding replication and uncovering therapeutic targets is critical for limiting disease in carriers.

In some embodiments, systems and methods of the invention target the HBV genome by finding a nucleotide string within a feature such as PreS1.

Guide RNA against PreS1 locates at the 5′ end of the coding sequence. Thus it is a good candidate for targeting because it represents one of the 5′-most targets in the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end.

The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation.

Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces. In some embodiments, method of the invention include creating one or several guide RNAs against key features within a genome such as the HBV genome. To achieve the CRISPR activity in cells, expression plasmids coding cas9 and guide RNAs are delivered to cells of interest (e.g., cells carrying HBV DNA). To demonstrate in an in vitro assay, anti-HBV effect may be evaluated by monitoring cell proliferation, growth, and morphology as well as analyzing DNA integrity and HBV DNA load in the cells.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1: Digesting Viral Nucleic Acid I

Methods and materials of the present invention may be used to digest foreign nucleic acid such as a genome of a hepatitis B virus (HBV).

It may be preferable to receive annotations for the HBV genome (i.e., that identify important features of the genome) and choose a candidate for targeting by enzymatic degredation that lies within one of those features, such as a viral replication origin, a terminal repeat, a replication factor binding site, a promoter, a coding sequence, and a repetitive region.

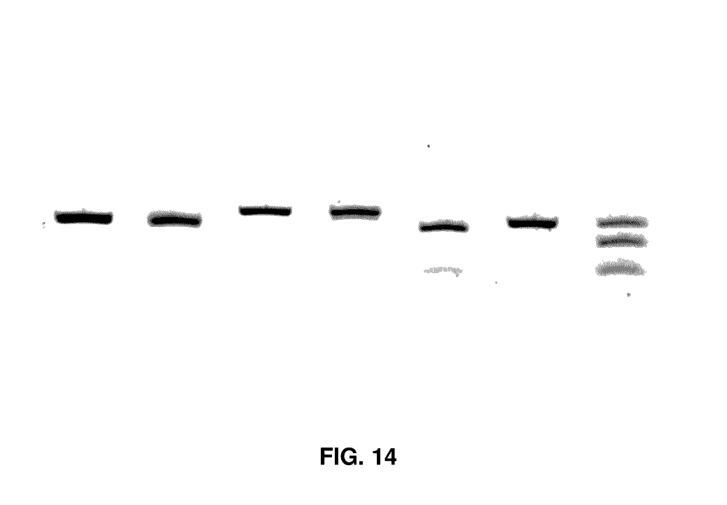

The use of Cas9 may be validated using an in vitro assay. To demonstrate, an in vitro assay is performed with cas9 protein and DNA amplicons flanking the target regions. Here, the target is amplified and the amplicons are incubated with cas9 and a gRNA having the selected nucleotide sequence for targeting. As shown in FIG. 14, DNA electrophoresis shows strong digestion at the target sites.

FIG. 14 shows a gel resulting from an in vitro CRISPR assay against HBV. Lanes 1, 3, and 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1. The presence of multiple fragments especially visible in lanes 5 and 7 show that sgHBV-Core and sgHBV-PreS1 provide especially attractive targets in the context of HBV and that use of systems and methods of the invention may be shown to be effective by an in vitro validation assay.

FIG. 14 gives results of digesting foreign nucleic acid. The nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even with repair mechanisms, the genome is render incapacitated.

### Example 2: Digesting Viral Nucleic Acid II

An exemplary assay shows the digestion of viral nucleic acid.

Burkitt's lymphoma cell lines Raji, Namalwa, and DG-75 were obtained from ATCC and cultured in RPMI 1640 supplemented with 10% FBS and PSA, following ATCC recommendation. Human primary lung fibroblast IMR-90 was obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven Cas9 were obtained from addgene, as described by Cong L et al. (2013) Multiplex Genome Engineering Using CRISPR/Cas Systems. Science 339:819-823.

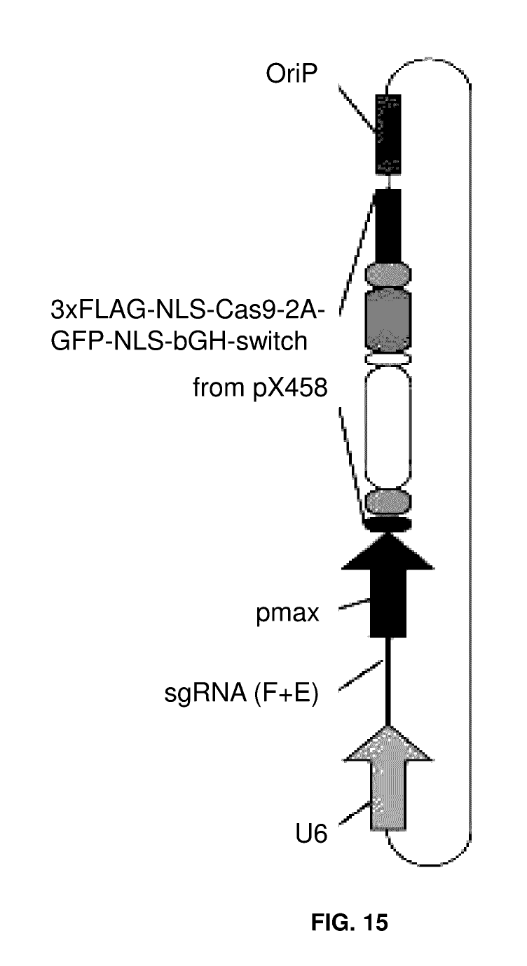

FIG. 15 shows a plasmid according to certain embodiments. An EGFP marker fused after the Cas9 protein allowed selection of Cas9-positive cells. A modified chimeric guide RNA stem-loop design was adapted for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

We obtained pX458 from Addgene, Inc. A modified CMV promoter with a synthetic intron (pmax) was PCR amplified from Lonza control plasmid pmax-GFP. A modified guide RNA sgRNA(F+E) was ordered from IDT. EBV replication origin oriP was PCR amplified from B95-8 transformed lymphoblastoid cell line GM12891. We used standard cloning protocols to clone pmax, sgRNA(F+E) and oriP to pX458, to replace the original CAG promoter, sgRNA and fl origin. We designed EBV sgRNA based on the B95-8 reference, and ordered DNA oligos from IDT. The original sgRNA place holder in pX458 serves as the negative control.

Lymphocytes are known for being resistant to lipofection, and therefore we used nucleofection for DNA delivery into Raji cells. We chose the Lonza pmax promoter to drive Cas9 expression as it offered strong expression within Raji cells. We used the Lonza Nucleofector II for DNA delivery. 5 million Raji or DG-75 cells were transfected with 5 ug plasmids in each 100-ul reaction. Cell line Kit V and program M-013 were used following Lonza recommendation. For IMR-90, 1 million cells were transfected with 5 ug plasmids in 100 ul Solution V, with program T-030 or X-005. 24 hours after nucleofection, we observed obvious EGFP signals from a small proportion of cells through fluorescent microscopy. The EGFP-positive cell population decreased dramatically after that, however, and we measured <10% transfection efficiency 48 hours after nucleofection. We attributed this transfection efficiency decrease to the plasmid dilution with cell division. To actively maintain the plasmid level within the host cells, we redesigned the CRISPR plasmid to include the EBV origin of replication sequence, oriP. With plasmid replication in the cells, the transfection efficiency rose to >60%.

To design guide RNA targeting the EBV genome, we relied on the EBV reference genome from strain B95-8.

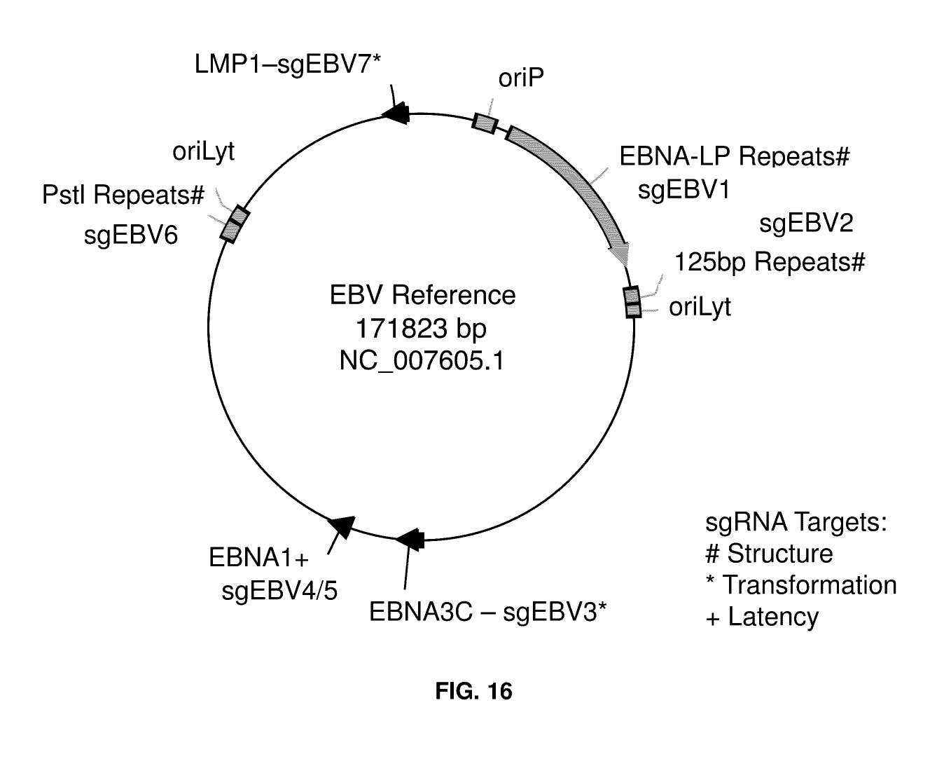

FIG. 16 diagrams the EBV genome. We targeted six regions with seven guide RNA designs for different genome editing purposes. The guide RNAs are listed in Table S1 in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

EBNA1 is crucial for many EBV functions including gene regulation and latent genome replication. We targeted guide RNA sgEBV4 and sgEBV5 to both ends of the EBNA1 coding region in order to excise this whole region of the genome. Guide RNAs sgEBV1, 2 and 6 fall in repeat regions, so that the success rate of at least one CRISPR cut is multiplied. These “structural” targets enable systematic digestion of the EBV genome into smaller pieces. EBNA3C and LMP1 are essential for host cell transformation, and we designed guide RNAs sgEBV3 and sgEBV7 to target the 5′ exons of these two proteins respectively.

EBV Genome Editing.

The double-strand DNA breaks generated by CRISPR are repaired with small deletions. These deletions will disrupt the protein coding and hence create knockout effects. SURVEYOR assays confirmed efficient editing of individual sites. Beyond the independent small deletions induced by each guide RNA, large deletions between targeting sites can systematically destroy the EBV genome.

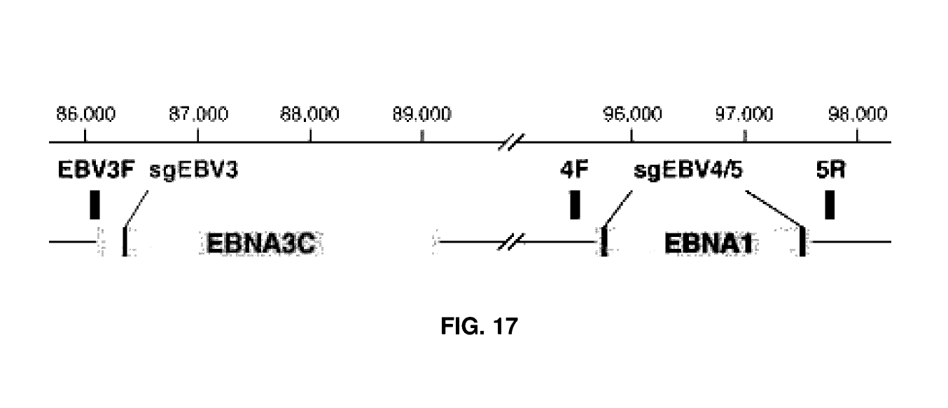

FIG. 17 shows genomic context around guide RNA sgEBV2 and PCR primer locations.

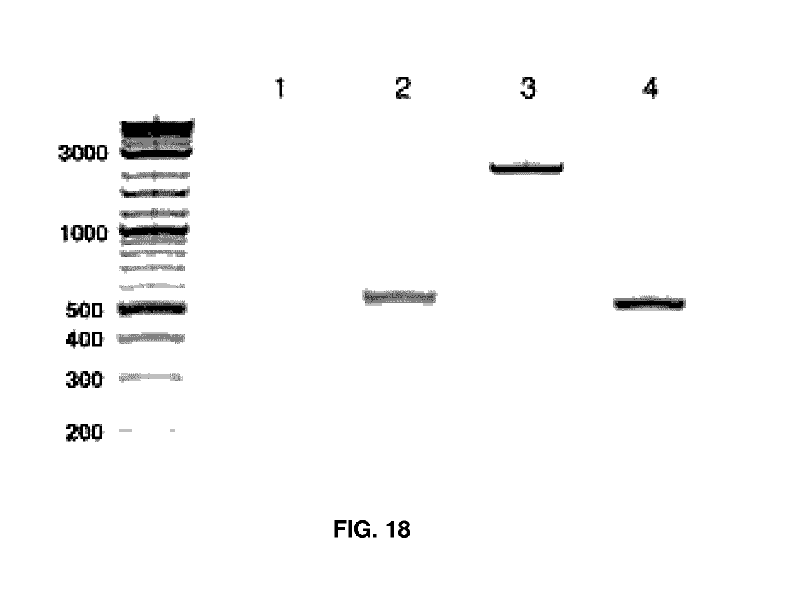

FIG. 18 shows a large deletion induced by sgEBV2, where lane 1-3 are before, 5 days after, and 7 days after sgEBV2 treatment, respectively. Guide RNA sgEBV2 targets a region with twelve 125-bp repeat units (FIG. 8). PCR amplicon of the whole repeat region gave a ˜1.8-kb band (FIG. 16). After 5 or 7 days of sgEBV2 transfection, we obtained ˜0.4-kb bands from the same PCR amplification (FIG. 16). The ˜1.4-kb deletion is the expected product of repair ligation between cuts in the first and the last repeat unit (FIG. 15).

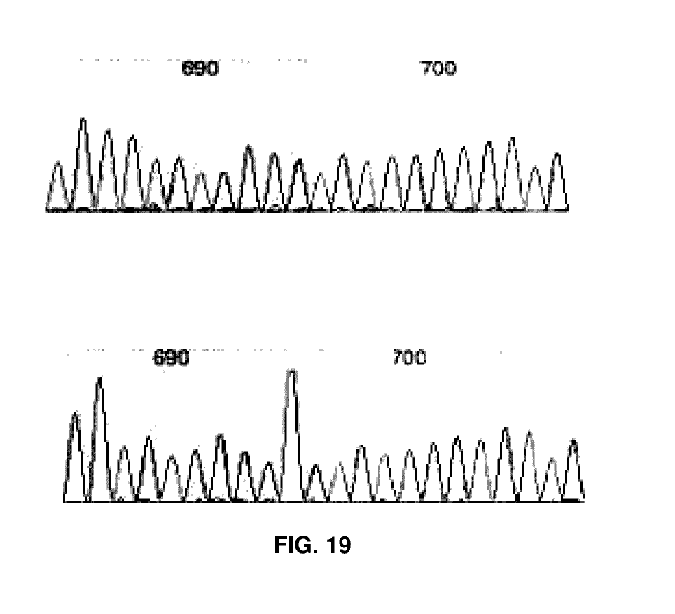

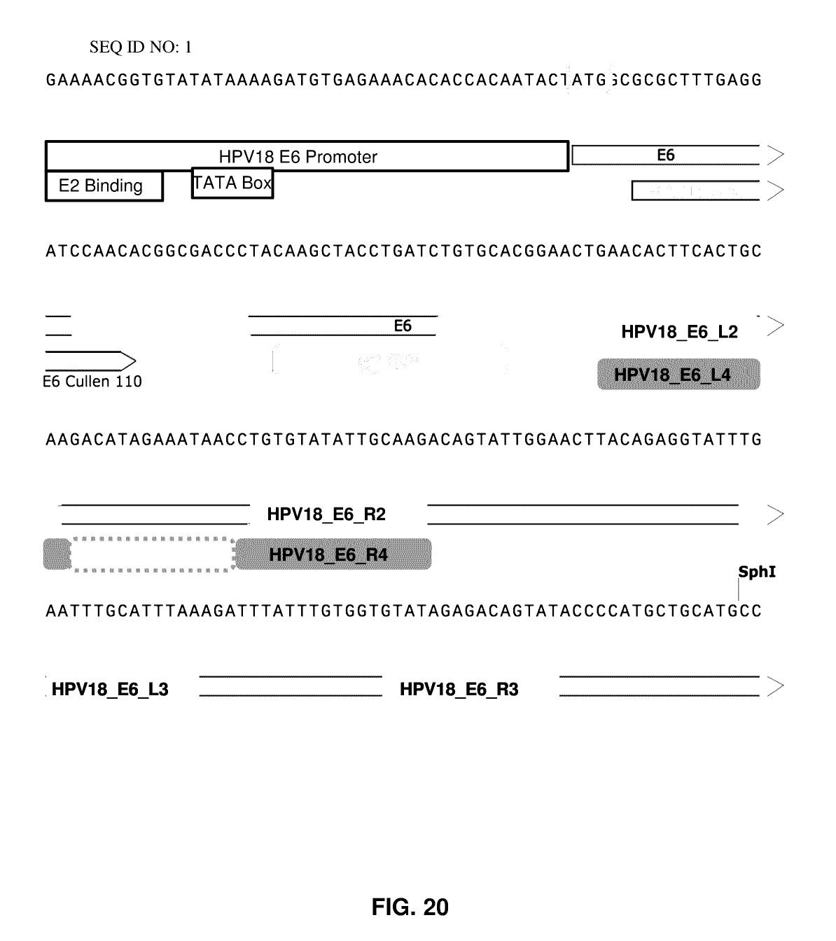

DNA sequences flanking sgRNA targets were PCR amplified with Phusion DNA polymerase. SURVEYOR assays were performed following manufacturer's instruction. DNA amplicons with large deletions were TOPO cloned and single colonies were used for Sanger sequencing. EBV load was measured with Taqman digital PCR on Fluidigm BioMark. A Taqman assay targeting a conserved human locus was used for human DNA normalization. 1 ng of single-cell whole-genome amplification products from Fluidigm C1 were used for EBV quantitative PCR. We further demonstrated that it is possible to delete regions between unique targets (FIG. 10). Six days after sgEBV4-5 transfection, PCR amplification of the whole flanking region (with primers EBV4F and 5R) returned a shorter amplicon, together with a much fainter band of the expected 2 kb (FIG. 20).

FIG. 19 shows that Sanger sequencing of amplicon clones confirmed the direct connection of the two expected cutting sites. A similar experiment with sgEBV3-5 also returned an even larger deletion, from EBNA3C to EBNA1. Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

Essential Targets For EBV Treatment. The seven guide RNAs in our CRISPR cocktail target three different categories of sequences which are important for EBV genome structure, host cell transformation, and infection latency, respectively. To understand the most essential targets for effective EBV treatment, we transfected Raji cells with subsets of guide RNAs. Although sgEBV4/5 reduced the EBV genome by 85%, they could not suppress cell proliferation as effectively as the full cocktail. Guide RNAs targeting the structural sequences (sgEBV1/2/6) could stop cell proliferation completely, despite not eliminating the full EBV load (26% decrease). We conclude that systematic destruction of EBV genome structure appears to be more effective than targeting specific key proteins for EBV treatment.

As noted, switched nucleic acids may encode a nuclease such as a TALEN (GenBank accession number: X05015.1) along with various HPV 18 TALENs designed to bind multiple E6 gene segments. The E6 gene is required for cell transformation and ongoing replication. Pairs of TALENs comprising HPV18_E6_L1 and R1, L2 and R2, L3 and R3, or L4 and R4 are shown.

FIG. 20 illustrates the HPV 18 E6 gene target sequence of a guide RNA (sgE6-2) for use with a guided nuclease such as Cas9 or dCas9. In various embodiments, nucleic acids encoding the TALENs or sgRNA depicted in FIG. 20 may include a riboswitch of the invention configured to allow translation of the nuclease only in the presence of, for instance, a certain viral nucleic acid.

The depicted portion of the HPV genome is

(SEQ ID NO.: 1)
GAAAACGGTG TATATAAAAG ATGTGAGAAA CACACCACAA
TACTATGGCG CGCTTTGAGG ATCCAACACG GCGACCCTAC
AAGCTACCTG ATCTGTGCAC GGAACTGAAC ACTTCACTGC
AAGACATAGA AATAACCTGT GTATATTGCA AGACAGTATT
GGAACTTACA GAGGTATTTG AATTTGCATT TAAAGATTTA
TTTGTGGTGT ATAGAGACAG TATACCCCAT GCTGCATGCC

### Example 3: HPV 18-Specific TALENs Shown to Kill HPV 18+ Cancer Cells

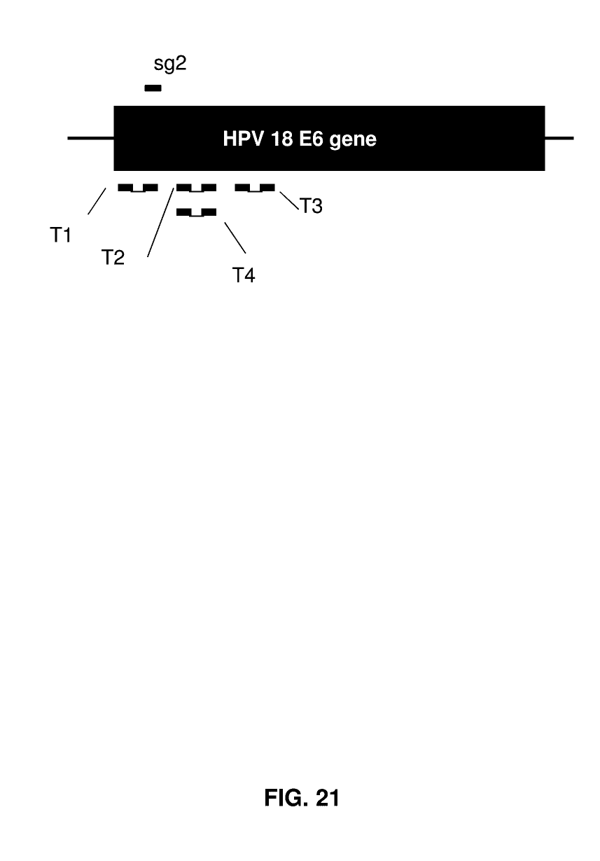

Switched nucleases of the invention may include TALENS or Cas9. TALENs with multiple binding domains have been shown to kill HPV 18+ cancer cells. Fusion polypeptides may be expressed in cells that have been transfected with plasmid DNA encoding the fusion polypeptide. HPV 18+ HeLa cells were plated and then transfected the next day with plasmid DNA complexed with cationic liposome. Plasmids encoding various TALENs were used included pAAVS1Talen1, pHPV18E6Talen1 (T1), pHPV18E6Talen2 (T2), pHPV18E6Talen3 (T3), pHPV18E6Talen4 (T4). Plasmids encoding the p113-HPV18E6-2-Cas9 (sg2) and p102-AAVS1-Cas9 complexes were also used. The targeted regions of the HPV 18 E6 gene are shown in FIG. 21.

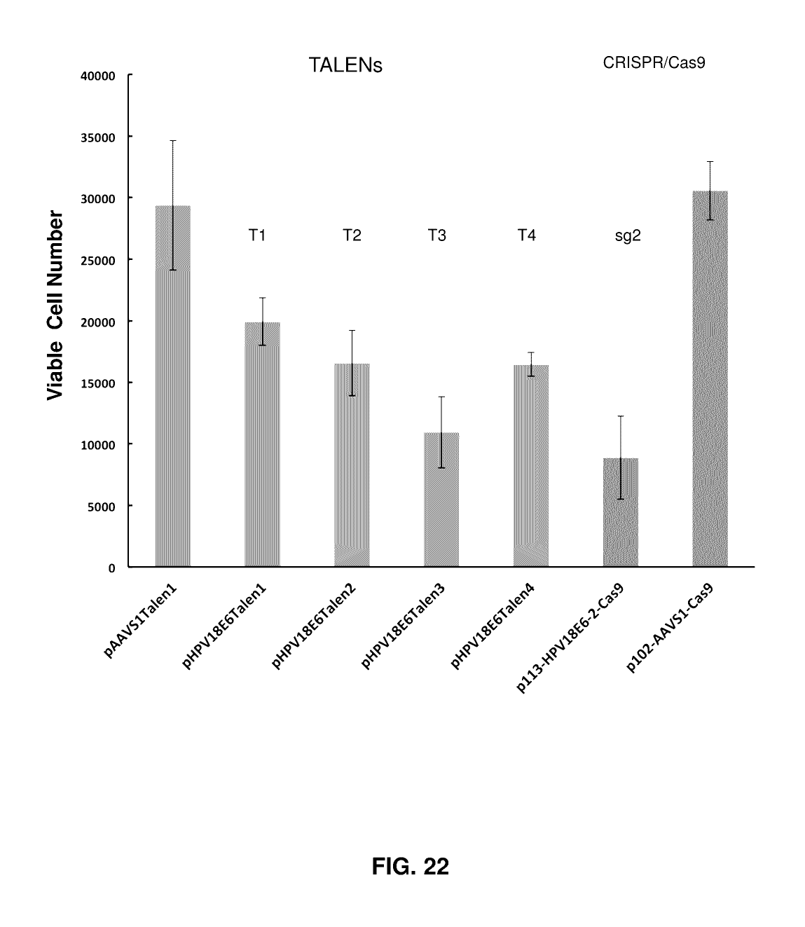

Viable cells were counted on day 5 for each of the transfected cell plates. Similar killing rates were observed with HPV 18 E6-specific TALEN (pHPV18E6Talen3) and CRISPR/Cas9 (p113-HPV18E6-2-Cas9). The viable cell counts for each of the TALENs and CRISPR/Cas9 complexes is shown in FIG. 22.

The AAVS1 site is present in the human genome and, as shown in FIG. 22, cleavage at AAVS1, unlike cleavage in the HPV 18 E6 region, does not kill cells as indicated by the increased cell counts on the plates containing cells transfected with pAAVS1Talen1 and p102-AAVS1-Cas9.

### Example 4

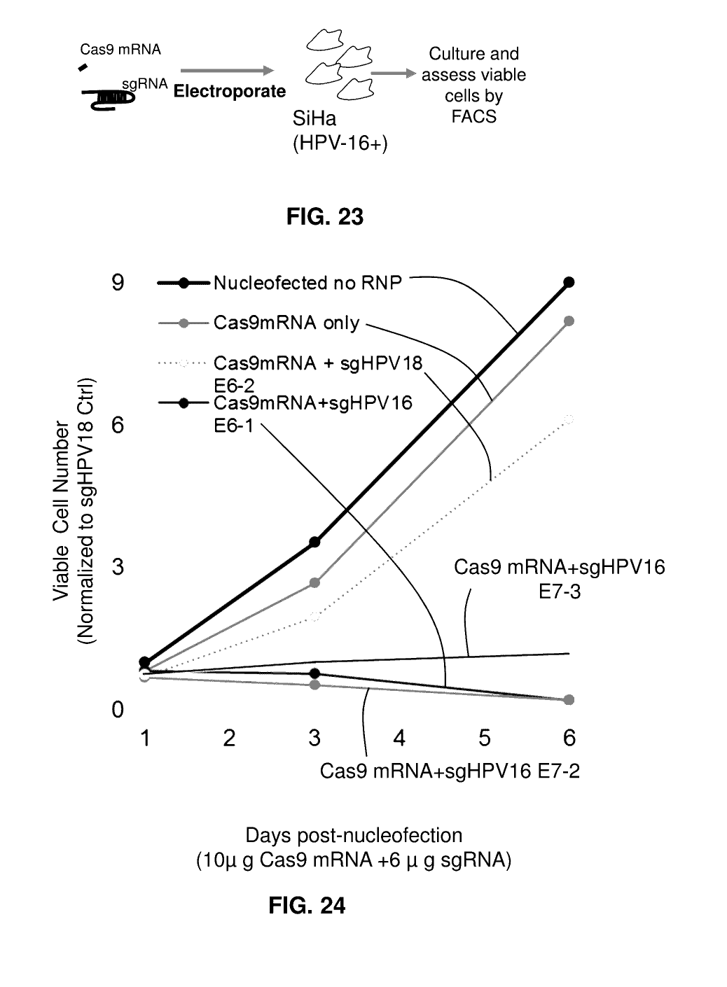

In various embodiments, switches of the invention may be tied to mRNA encoding an endonuclease such as Cas9. Switched mRNA may be introduced into a cell by electroration. Cas9 endonuclease has been found to kill HPV 16+ cancer cells by treating the HPV 16+ cancer cells through electroporation with HPV 16-specific sgRNA and Cas9 encoding mRNA. As illustrated in FIG. 23, an mRNA encoding Cas9 protein and an sgRNA were introduced into SiHa HPV-16+ cells through electroporation. The cells were then cultured and viable cell counts were taken using fluorescence-activated cell sorting (FACS).

FIG. 24 shows normalized cell counts after 1, 3, and 6 days post-nucleofection with the various Cas9 mRNA and sgRNA combinations, all normalized to the sgHPV18 control.

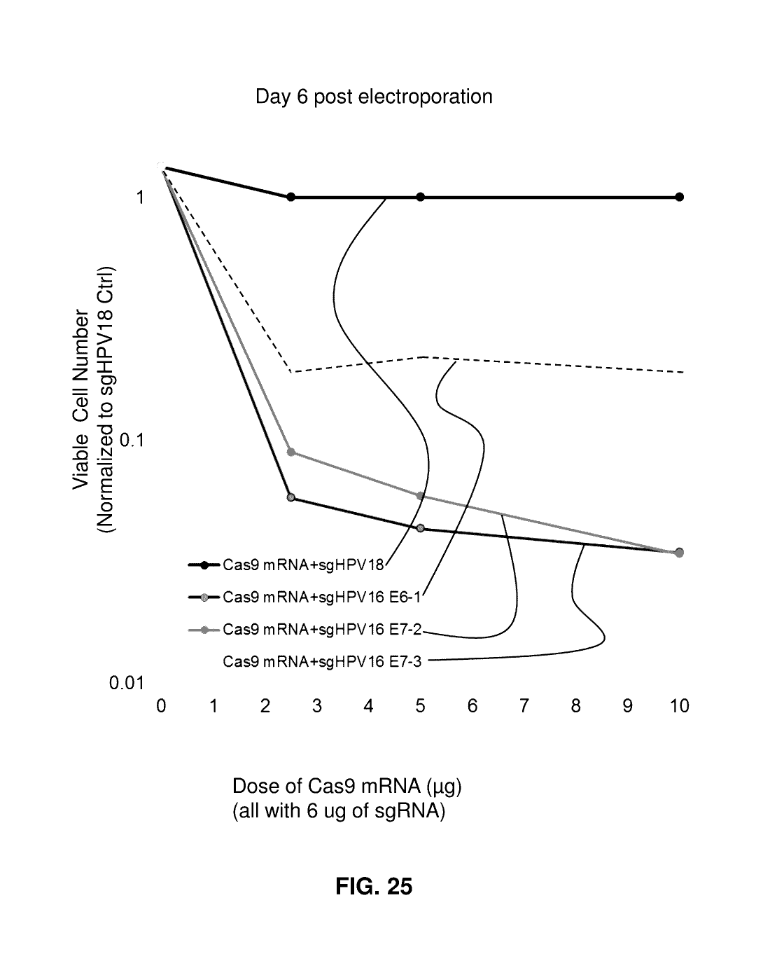

FIG. 25 shows cell counts for cells treated with 6 μg of the various sgRNA and a variety of Cas9 mRNA after 6 days, normalized to the sgHPV18 control. Both FIG. 24 and FIG. 25 show reduced cell counts in the cells nucleofected with HPV 16− specific sgRNAs and Cas9 mRna.

### Example 5

In certain embodiments, switched endonuclease encoding nucleic acids may be transduced into a target cell using viral vectors such as a lentiviral vector. Switched endonuclease may be used to target HBV nucleic acid in a host cell. Targeted Cas9 has been shown to cleave HBV DNA in an HBV episomal DNA cell model using lentivirus transduction.

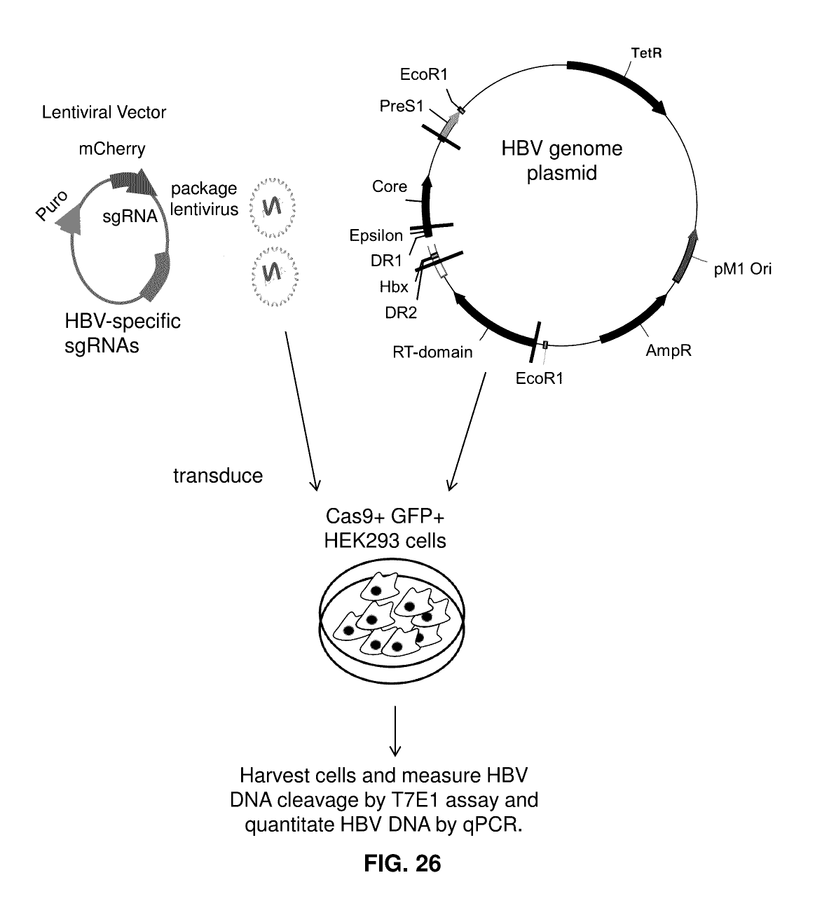

FIG. 26 illustrates an HBV episomal DNA cell model. Cas9+GFP+HED293 cells were transfected with an HBV genome plasmid as shown. HBV-specific sgRNAs were then introduced through transduction using a lentiviral vector. The cells were then harvested an HBV DNA cleavage was measured by T7E1 assay and HBV DNA was measured by qPCR.

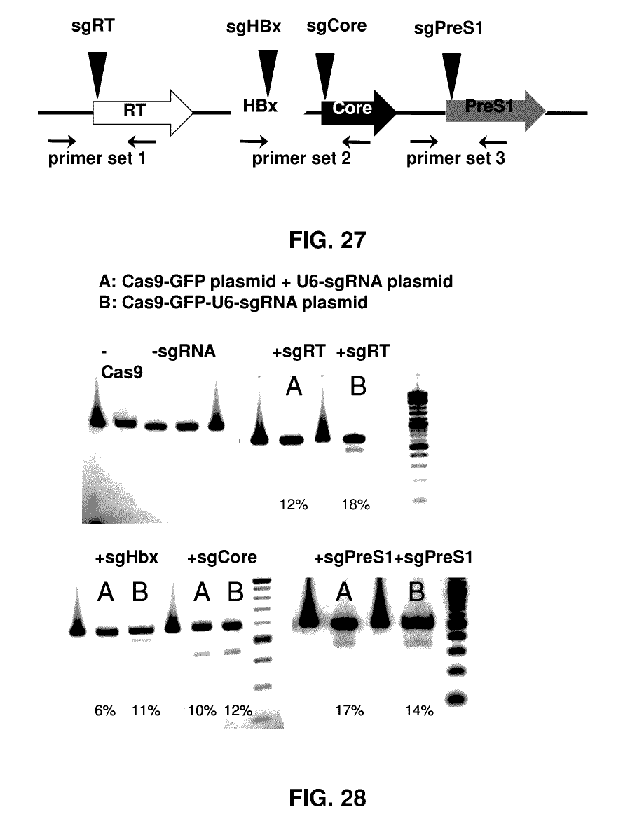

FIG. 27 shows the target locations on the HBV genome of various sgRNAs used in the model along with the location of primer set targets used to assess HBV DNA cleavage.

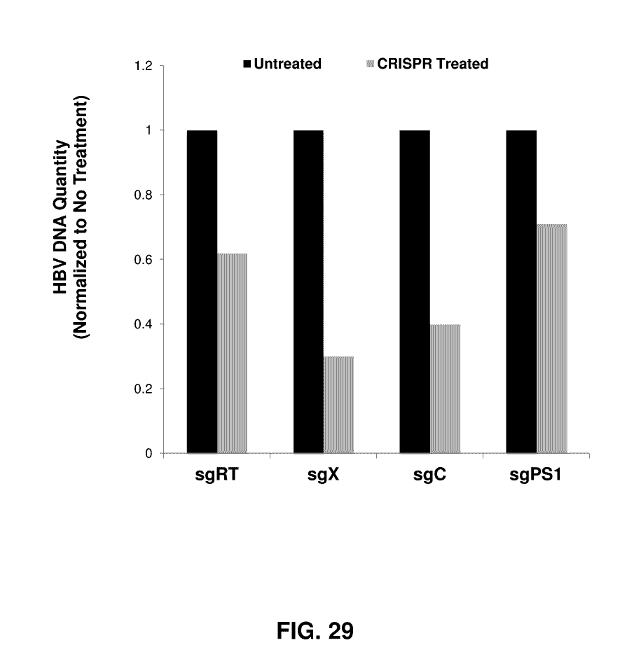

FIG. 28 shows the results of gel electrophoresis indicating cleavage of HBV DNA in cells transduced with sgRT RNA, sgHBx RNA, sgCore RNA, and sgPreS1 RNA. FIG. 29 shows HBV DNA quantity determined by qPCR in untreated cells and cells treated with HBV-specific sgRNAs and Cas9. Each of the four tested sgRNAs exhibited reduced HBV DNA quantity when compared to untreated cells. The results illustrated in FIGS. 28 and 29 are from unsorted cells 2 days post treatment.

What is claimed is:

1. A nucleic acid that encodes:
a nuclease; and
a switch that causes the nuclease to be expressed in the presence of a viral nucleic acid.

2. The nucleic acid of claim 1, wherein a portion of the switch is complementary to at least a portion of a latency associated transcript.

3. The nucleic acid of claim 2, wherein the latency associated transcript comprises one selected from the group consisting of: an HHV latency associated transcript; and a latency-associated transcript of pseudorabies virus.

4. The nucleic acid of claim 2, wherein the latency associated transcript when present interacts with the switch to initiate translation of the nuclease.

5. The nucleic acid of claim 4, wherein the nucleic acid is a plasmid.

6. The nucleic acid of claim 1, wherein the nucleic acid is mRNA comprising a 5′ cap and poly(A) tail.

7. The nucleic acid of claim 5, wherein the nuclease is Cas9 endonuclease.

8. The nucleic acid of claim 7, wherein the nucleic acid further encodes a guide sequence that targets the nuclease to a target on a genome of a virus.

9. The nucleic acid of claim 8, wherein the target comprises a segment of at least 18 nucleotides that is at least 60% complementary to the guide sequence and is adjacent a protospacer adjacent motif (PAM), and wherein the target is not found in the host genome.

10. The nucleic acid of claim 9, wherein the target in the viral genome includes a portion of a genome or gene of one selected from the group consisting of: a hepatitis virus; a hepatitis B virus (HBV); an Epstein-Barr virus; a Kaposi's sarcoma-associated herpesvirus (KSHV); a herpes-simplex virus (HSV); a cytomegalovirus (CMV); and a human papilloma virus (HPV).

11. The nucleic acid of claim 8, wherein the switch is a riboswitch.

12. The nucleic acid of claim 11, wherein the riboswitch is a portion of the nucleic acid that, when transcribed into mRNA, forms a double stranded structure that blocks translation in the absence of the viral nucleic acid.

13. The nucleic acid of claim 12, wherein the switch includes one or more of a ribosome binding site and a start codon.

14. The nucleic acid of claim 13, wherein when the plasmid is transcribed into RNA and the latency associated transcript hybridizes to the riboswitch, the Cas9 endonuclease is expressed.

15. The nucleic acid of claim 1, wherein the viral nucleic acid required for expression of the nuclease is a latency-associated transcript.

16. The nucleic acid of claim 15, wherein the nuclease is one selected from the group consisting of a zinc-finger nuclease, a transcription activator-like effector nuclease, and a meganuclease.

17. The nucleic acid of claim 1, wherein the switch causes translation of the nuclease upon hybridization of the viral nucleic acid to the switch.

18. The nucleic acid of claim 1, wherein the viral nucleic acid is from a virus selected from the group consisting of adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, and banna virus.


SEQUENCE LISTING (1 sequence)

SEQ ID NO: 1 - DNA, 240 bp, Human papillomavirus type 18
gaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgcc